SEQUENCE LISTING (27 sequences)

SEQ ID NO: 1 - DNA, 573 bp, Glycine max
atgcgttatttgtctctgagtactttttctctcttgtgcattacctttgtggtcatggcagcctccgtggatcccctagtggttggtcgcgtgatcggcgatgtggtagacatgttcattccttcagtcaacatgtccgtttactttgggtcgaagcacgtcacaaatggctgtgacatcaagccatccattgccatcagccctcctaagctcaccctcaccggcaacatggataacctctacacactggttatgactgatcctgacgcacctagccccagtgaaccaagcatgcgcgagtggatacattggatcttagttgacatacctggaggaacaaacccatttcgcggaaaagagattgtttcatatgtgggaccaagaccacctattggaatacatcgctatatctttgtgttgtttcaacagaaaggacctttaggtcttgtggagcaaccaccaactcgagcaagcttcaacactcgttattttgccaggcaattggacttgggacttccagtggccactgtctacttcaactctcaaaaagaacctgctgttaagaggcgctga

SEQ ID NO: 2 - protein, 190 aa, Glycine max
MRYLSLSTFSLLCITFVVMAASVDPLVVGRVIGDVVDMFIPSVNMSVYFGSKHVTNGCDIKPSIAISPPKLTLTGNMDNLYTLVMTDPDAPSPSEPSMREWIHWILVDIPGGTNPFRGKEIVSYVGPRPPIGIHRYIFVLFQQKGPLGLVEQPPTRASFNTRYFARQLDLGLPVATVYFNSQKEPAVKRR

SEQ ID NO: 3 - DNA, 573 bp, Glycine max
atgcgttatttgtctctgagtactttttctctcttgtgcattacctttgtggtcatggcagcctccgtggatcccctagtggttggtcgcgtgatcggcgatgtggtagacatgttcattccttcagtcaacatgtccgtttactttgggtcgaagcacgtcacaaatggctgtgacatcaagccatccattgccatcagccctcctaagctcaccctcaccggcaacatggataacctctacacactggttatgactgatcctgacgcacctagccccagtgaaccaagcatgcgcgagtggatacattggatcttagttgacatacctggaggaacaaacccatttcgcggaaaagagattgtttcatatgtgggaccaagaccacctattggaatacatcgctatatctttgtgtcctttcaacagaaaggacctttaggtcttgtggagcaaccaccaactcgagcaagcttcaacactcgttattttgccaggcaattggacttgggacttccagtggccactgtctacttcaactctcaaaaagaacctgctgttaagaggcgctga

SEQ ID NO: 4 - protein, 190 aa, Glycine max
MRYLSLSTFSLLCITFVVMAASVDPLVVGRVIGDVVDMFIPSVNMSVYFGSKHVTNGCDIKPSIAISPPKLTLTGNMDNLYTLVMTDPDAPSPSEPSMREWIHWILVDIPGGTNPFRGKEIVSYVGPRPPIGIHRYIFVSFQQKGPLGLVEQPPTRASFNTRYFARQLDLGLPVATVYFNSQKEPAVKRR

SEQ ID NO: 5 - DNA, 573 bp, Glycine soja
atgcgttatttgtctctgagtactttttctctcttgtgcattacctttgtggtcatggcagcctccgtggatcccctagtggttggtcgcgtgatcggcgatgtggtagacatgttcattccttctgtcaacatgtccgtttactttgggtcgaagcacgtcacaaatggctgtgacatcaagccatccatcgccatcagccctcccaagctcaccctcaccggcaacatggataacctctacacactggttatgactgatcctgacgcacctagccccagtgaaccaagcatgcgtgagtggatacattggatcgtagttgacatacctggaggaacaaacccatttcgcggaaaagagattgtttcatatgtgggaccaaggccacctattggaatacatcgctatatctttgtgttgtttcaacagaaaggacctttaggtcttgtggagcaaccaccaactcgtgcaagcttcaacactcgttattttgccaggcaattggacttgggacttccagtggccactgtctacttcaactctcaaaaagaacccgctgttaagaggcgctga

SEQ ID NO: 6 - protein, 190 aa, Glycine soja
MRYLSLSTFSLLCITFVVMAASVDPLVVGRVIGDVVDMFIPSVNMSVYFGSKHVTNGCDIKPSIAISPPKLTLTGNMDNLYTLVMTDPDAPSPSEPSMREWIHWIVVDIPGGTNPFRGKEIVSYVGPRPPIGIHRYIFVLFQQKGPLGLVEQPPTRASFNTRYFARQLDLGLPVATVYFNSQKEPAVKRR

SEQ ID NO: 7 - DNA, 3468 bp, Glycine max
tgtatttgatagtttaattttattttaaaattaaaattcataaataaaataaatgactgcatagtaaataatatataaccaatgcaggaattcaaatactatatgagcaaacaattaaaacttggtatcctctctcctaattttttaattgtatatctaagtctctaatttgagactacaagttaaattagaaaaattttatactaattgatttaggtaaataatggtaattgtctaaataagtaatacaatttataaaataaataagaaaaaaaatgaagtgcatccctagttttatattacaaataatgataaaattgtttattattctacattgtataaaacaataagtttttaaaaaattataaacttaaattcattaaatatacaataaaagaatataatactttatataatgtttttattttcattattattattacgtcacccattttatatgtttttgtgagtataatctgtccaaaaatacattgaatggaaggcaataaatatccacttaaaataatattttttaagacaaaataacatttattttatcttttctcttttttatttatatggattctatatcattttgtgtggatataatttgtctaaaaatataattaatgaatgaaagataataatgatccacttaaaataatattctcaaaggtaacataatttttttatattttattttcttaattaaatagaaatatatattttttgtgtgaatataatttgtttaaaacatattgaatgagaaaccataattaatgatccacttaaaataatattttttaaggtaaaataatattactccagtcacctctaacatattatgaagtatttcttttattttgagagctggggataatatctctctgtcaatacatgcatccaattacctttaacagagtgacctcataagtcatttattttatgtctttaatgagtgttaactttaactccactgaagaatcttccacgtcatccatttatgaaaccctttaaacagccatggccccacctcactatagacaaacaaggaactgtgggagttacgaaaagaggcattgttctcctactgcagaaaccaccattgcaatttgcaccctcattctcacgttacacacactctgcttatattgtagagcaagcatggcttgaagtttgaacccatttactgtttttctgatgcagttagtttcaaatgggcggctttgatatggccatttgtcacaaagaaacagaagatgactgatttactctctgcaaatccaccaacgtgtcatccattgcatgtccctgattttacgatggtccatgcatgatcacaaaattacaaaatagaggtccctgatattttacccttcagtttttttcttttctttttgagctatataaaccctaccctatacactgtaagcaaccaccagaaactctcactagcttagtttctatatatgcgttatttgtctctgagtactttttctctcttgtgcattacctttgtggtcatggcagcctccgtggatcccctagtggttggtcgcgtgatcggcgatgtggtagacatgttcattccttcagtcaacatgtccgtttactttgggtcgaagcacgtcacaaatggctgtgacatcaagccatccattgccatcagccctcctaagctcaccctcaccggcaacatggataacctctacacactggtattcattcatgtttctgttgagagatctcacatgcatgcatcaaggtttttcttaattttatgctttctctttttctttaatgtgcctacaggttatgactgatcctgacgcacctagccccagtgaaccaagcatgcgcgagtggatacattggttagtaaatttgttttagttcttcctattcttagtttctaactccatatatataaactgatatgatcttgctgtttccacttattttccaggatcttagttgacatacctggaggaacaaacccatttcgcggtagtaaaagaaatcttttggtctacttcatatatgacatttttattttcttctatttataaaagtttcatgatgttgcatgcatgagtactactgggttcagttctacttaattaatttttcttttcttcgggctaaaacatcatgttttttaattttttgggcgtccatcgcactgactctcgatttcagaaacatttttcttcactggtcataaaaatcttaggccatttctggcaaaatcaacatgcatgttcctacctaggctcctcgcatgcatgtttcagatttcttctggaaaaaaaaatcacagaatttcagggctatgtcacatcttgtgtgcacactctctttctatacatcaaatggtactctcacacctgtgatctaccaaaatttggatccaatgtccaccatcccccacgtgtcctgcttgtgcctgctatgaacacgtgtcacaacaaatattccatctctaaattctctgaaataatcgttaaataattgttaagtatagtaaataaataaattctcaattattctgtcatattttctctaaaaccattcttcaataaattaattttagattttctccgtgcatgcaccaatttagataacatgtgttagctagggaatctccttttatgagatcatcacacgtgccccatagcttgcatgttatcaaatttcagaattccatcacaagcaaatgtgtcaaacatgaagcaatatttctccaactccaaagtaaaaatattttgaattatcacctttctttggtcctgtcatttctaaaatataatcttcgcatctagcatacaggatattagattaatttttcctactttcaacagttaacattgatttcttgtaaacaataataatttgattcatgcggaacaaagaaatctaataattaatcaagtaaatggtcttctttgcagggaaacaaagaaatctagtaattaatcaagtaaattgtcttctttgcaggaaaagagattgtttcatatgtgggaccaagaccacctattggaatacatcgctatatctttgtgttgtttcaacagaaaggacctttaggtcttgtggagcaaccaccaactcgagcaagcttcaacactcgttattttgccaggcaattggacttgggacttccagtggccactgtctacttcaactctcaaaaagaacctgctgttaagaggcgctgaatctagctatattgtaaccatcagtgtctctcttgagatatgcatggttggaatatacttttaagatatcagaactatataaaatctatgactagtgcgtaaataatgcagggagagggtgtgtgtaaagtaatatagttgtccaagacatgagtggtgccgaccatttccccggctttgataactttttttctctatatatatacctctctttcactctatcaaatatataaagttaatctttatt

SEQ ID NO: 8 - DNA, 3475 bp, Glycine soja
tgtatttgatagtttaattttattttaaaattaaaattcataaataaaataaatgagtgcatagtaaataatatataaccaatgcaggaattcaaatactatatgagcaaacaattaaaacttggtatcctctctcctaatttgttaattgtatatctaagtctctaatttgagactacaagttaaattagaaaaaatttatactaattgatttaggtaaataatggtaattgtctaaataagtaatacaatttataaaataaataagaaaaaaaatgaagtgcatccctagttatagatagatatttaatttttatattacaaataatgataaaattgtttattattctacatagtataaaacaataagtttttataaaattataaacttaaattcattaaatatacaataaaagaatataatactttatataatgtttttattttcattattattattacgttacccattttatatgtttttgtgagtataatctgtccaaaaatacattgaatggaaggcaataaatatccacttaaaataatattttttaagacaaaataacatttattttatcttttctctttcttatttatatggattctatatcattttgtgtggatataatttgtctaaaaatataattaatgaatgaaagataataatgatccacttaaaataatattttcaaagataacataatttttttatattttattttcttaattaaatagaaatatatattttttgtgaatataatttgtttaaaacatattgaatgagaaaccataatattttttaaggtaaaataacatcactccagtcacctctaacatattatggagtatttcttttattttgagagctggggataatatctctctgtcaatacatgcatccaattacctttaacagagtgacctcataagtcatttattttatgtctttaatgagtgttaactttaactccactgaagaatcttccacgtcatccatttatgaaaccctttaaacagccatggccccacctcactatcgacaaacaaggaactgtgggagttacgaagagaggcagtgttctcctactgcagaaaccaccattgcaatttgcaccctcattctcacgttacacacactctgcttatattgtagcgcaagcatggcttgaagtttgaacccatttactgtttttctgatgcagttagtttcaaatgggcggctttgatatggccatttgtcacaaagaaacagaagatgactgatttactctctgcaaatccaccaacgtgtcatccattgcatgtccctgattttacgatggtccatgcatgatcacaaaattacaaaatagaggtccctgatattttacccttcagtttttttcttttctttttgagctatataaaccctaccctatacactgtaagcaaccaccagaaactctcactagcttagtttctatatatgcgttatttgtctctgagtactttttctctcttgtgcattacctttgtggtcatggcagcctccgtggatcccctagtggttggtcgcgtgatcggcgatgtggtagacatgttcattccttctgtcaacatgtccgtttactttgggtcgaagcacgtcacaaatggctgtgacatcaagccatccatcgccatcagccctcccaagctcaccctcaccggcaacatggataacctctacacactggtattaattcatgtttctgttgagagatctcacatgcatgcatcaaggtttttcttaattttatgctttctctttttctttaatgtgcctacaggttatgactgatcctgacgcacctagccccagtgaaccaagcatgcgtgagtggatacattggttagtaaatttgttttagttcttcctattcttagtttctaactctatatatataaactgatatgatcttgctgtttacacttattttccaggatcgtagttgacatacctggaggaacaaacccatttcgcggtagtaaaagaaatcttttggtctacttcatatatgacatttttattttcttctatttataaaagtttcatgatgttgcatgcatgagtactactgggttcagttctacttaattaatttttcttttcttcgggctaaaacatcatgttttttaatttcttgggcgtccattgcactgactctcagatttcagaaacatttttcttcactggtcataaaaatcttaggccatttctggcaaaatcaacatgcatgttcctacctaggctcctcgcatgcatgtttcagatttcttctggaaaaaaaaatcacagaatttcagggctatgtcacatcttgtgtgcacactctctttctatacatcaaatggtactctcacacctgtgatctaccaaaatttggatccaatgtccaccatcccccacgtgtcctgcttgtgcctgctatgaacacgtgtcacaacaaatattccatctctaaattctctgaaataatcgttaaataattgttaagtatagtaaataaataaattctcaattattctgtcatattttctctaaaaccattcttcaataaattaattttagattttctcagtgcatgcaccaatttagataacatgtgttagctagggaatctccttttatgagatcatcacacgtgccccatagcttgcatgttatcaaatttcagaattccatcacaaatgtgtcaaacatgaaacaatatttctccaactccaaagtaaatattttgaattatcacctttctttggtcctgtcatgtctaaaatataatcttcacatctagcatataggatattagattaatttttcctactttcaatagttaacgttgatttcttgtaaacaataataatttgattcatgcagaacaaatctaataattaaagtaaatggtcttctttgcagggaaacaaagaaatctagtaattaatcaagtaaattgtcttctttgcaggaaaagagattgtttcatatgtgggaccaaggccacctattggaatacatcgctatatctttgtgttgtttcaacagaaaggacctttaggtcttgtggagcaaccaccaactcgtgcaagcttcaacactcgttattttgccaggcaattggacttgggacttccagtggccactgtctacttcaactctcaaaaagaacccgctgttaagaggcgctgaatctagctgttaagaggcgctgaatctagctatattgtaaccatcagtgtctctcttgagatatgcatggttggaatatactttaagatatcagaactatataaaatctatgactagtgcgtaaataatgcagggagagggtgtgtgtaaagtaatatagttgtccaagacctgagtggtgccgaccatttccccggctttgataactttttttctctatatatatacctctctttcactctatcaaatatataaagttaatctttatta

SEQ ID NO: 9 - DNA, 522 bp, Brassica napus
atggcagcttcggttgatcctttggtggttggaagagtgatcggagatgtgttggacatgttcatccccaccgccaacatgtctgtctactttggccccaaacacataactaacggctgcgagatcaaaccctctgccgcagtcaaccctccaaaagtcaacatctccggcaactccaatgagctttacactctcgtgatgactgacccggacgcacctagcccgagtgagccgaacatgagagaatgggtccattggattgtcgtggatatcccgggaggcaccaacccctcaaaaggaaaggagatactgccatatatggagccgagaccaccggtggggattcaccgttacatatttgtacttttcaggcagaactcaccggtgggtatgatggtgcagcagccgccttcgcgagccaacttcagcacccgaatgttcgctggacatctcgatcttggtttgcctgtggccacagtttacttcaacgcccagaaagagccagcttcacgcagacgctga

SEQ ID NO: 10 - protein, 173 aa, Brassica napus
MAASVDPLVVGRVIGDVLDMFIPTANMSVYFGPKHITNGCEIKPSAAVNPPKVNISGNSNELYTLVMTDPDAPSPSEPNMREWVHWIVVDIPGGTNPSKGKEILPYMEPRPPVGIHRYIFVLFRQNSPVGMMVQQPPSRANFSTRMFAGHLDLGLPVATVYFNAQKEPASRRR

SEQ ID NO: 11 - DNA, 519 bp, Gossypium raimondii
atggctgcctccgttgatcctctcgttgttggtcgtgtcattggcgatgttgtcgacatgttcgttcccaccgtcaccatgtccgtctactatggttccaagcacgtctctaacggatgtgacattaagccttctatggctatcaaccctcccaaagttgccatcgatggccttcctgaccagttttatactttggtgatgactgaccctgatgcgccaagtcccagcgagccaaccatgcgtgaatgggttcactggatcgtgtctgacattcctggaggaaccaaccccactcgagggaaggaaatcctggcatacatgggtccacggcctccagtaggcatacatcgctacatactggtgctgttccaacaaaagggcccgttgggtgcggtgcagcaaccggcgacaagagctaacttcagcactcgatttttcgctgaccacctcaacttgggactgcctgtggctactgtctatttcaacgcccaaaaagagcccgtcagccgaaggcgttga

SEQ ID NO: 12 - protein, 172 aa, Gossypium raimondii
MAASVDPLVVGRVIGDVVDMFVPTVTMSVYYGSKHVSNGCDIKPSMAINPPKVAIDGLPDQFYTLVMTDPDAPSPSEPTMREWVHWIVSDIPGGTNPTRGKEILAYMGPRPPVGIHRYILVLFQQKGPLGAVQQPATRANFSTRFFADHLNLGLPVATVYFNAQKEPVSRRR

SEQ ID NO: 13 - DNA, 519 bp, Zea maize
atggcgcgcttcgtggatccgctggtggtggggcgggtgatcggcgaggtggtggacctgttcgtgccttccatctccatgaccgtcgcctatgatggccccaaggacatcagcaacggctgcctcctcaagccgtccgccaccgccgcgccgccgctcgtccgcatctccggccgccgcaacgacctctacacgctgatcatgacggaccccgatgcgcctagccccagcaacccgaccatgagggagtacctccactggatagtgattaacataccaggaggaacagatgctactaaaggtgaggaggtggtggagtacatgggcccgcggccgccggtgggtatccaccgctacgtgctggtgctgttcgagcagaagacgcgcgtgcacgcggaggcccccggcgaccgcgccaacttcaagacgcgcgcgttcgcggcggcgcacgagctcggcctccccactgccgtcgtctacttcaacgcgcagaaggagcccgccagccgccgccgctag

SEQ ID NO: 14 - protein, 172 aa, Zea maize
MARFVDPLVVGRVIGEVVDLFVPSISMTVAYDGPKDISNGCLLKPSATAAPPLVRISGRRNDLYTLIMTDPDAPSPSNPTMREYLHWIVINIPGGTDATKGEEVVEYMGPRPPVGIHRYVLVLFEQKTRVHAEAPGDRANFKTRAFAAAHELGLPTAVVYFNAQKEPASRRR

SEQ ID NO: 15 - DNA, 543 bp, Triticum asestivum
atggcagcccatgtggatccccttgtggttgggagggtgatcggtgacgtggtggacatgttcgtgcccaccatgccggtgaccgtgcgcttcgggacgaaggacctgacgaacggctgcgagatcaagccgtccatcgccgacgcggcgccctcgatccagatagccggccgggccggcgatctcttcaccctggttatgactgatccggacgcaccgagccccagcgagccaaccatgaaggagtggcttcactggctggtggttaacatacctggtggatcagatccttctcaaggggaggaggtggtgccctacatgggtccgaagccgccgttgggcatccaccgctacgtgctggtgctgttccagcagaaggcgcgtgtgctggcgccggctcccggcggcgacacagcagcgtcggccatgcgcgcgcggttcagcacccgtgccttcgcagagcgccatgacctggggctccccgtcgccgccatgtacttcaacgcgcagaaggagccggccaaccgccgccgccgctactag

SEQ ID NO: 16 - protein, 180 aa, Triticum asestivum
MAAHVDPLVVGRVIGDVVDMFVPTMPVTVRFGTKDLTNGCEIKPSIADAAPSIQIAGRAGDLFTLVMTDPDAPSPSEPTMKEWLHWLVVNIPGGSDPSQGEEVVPYMGPKPPLGIHRYVLVLFQQKARVLAPAPGGDTAASAMRARFSTRAFAERHDLGLPVAAMYFNAQKEPANRRRRY

SEQ ID NO: 17 - DNA, 522 bp, Medicago truncatula
atgatggctgcctcggttgatcctttggtggttggtcgtgtgattggtgatgttgttgacatgttcattccatctgttggcatgtctgtttactttggtcctaaacatgtcacaaatggttgtgacataaagccatccatggctatcaacccacccaaggtcactctcactggaaacatggataacctctacactctggttatgactgatccagatgcaccaagccccagtgagccaagcatgcgtgaactgatacactggattgtggttgacattcctggaggaacaaatccaaagcgaggaaaggagattcttccatatataggaccaaaaccaccggttggtatccaccgatacatattggttttgtttgagcagaagggaccaattggaatggttgagcagccaaccagtagagtcagtttcaacactcgttattttgcaagtcaaatgaaccttggtcttcctgtagctacagtctactttaactctcagaaggagcctcaggctaagaggcgttga

SEQ ID NO: 18 - protein, 173 aa, Medicago truncatula
MMAASVDPLVVGRVIGDVVDMFIPSVGMSVYFGPKHVTNGCDIKPSMAINPPKVTLTGNMDNLYTLVMTDPDAPSPSEPSMRELIHWIVVDIPGGTNPKRGKEILPYIGPKPPVGIHRYILVLFEQKGPIGMVEQPTSRVSFNTRYFASQMNLGLPVATVYFNSQKEPQAKRR

SEQ ID NO: 19 - DNA, 531 bp, Oryza sativa
atggcatcgcatgtggacccgctggtggtggggagggtgatcggcgacgtggtggacctgttcgtgccgacgacggccatgtcggtgcggttcgggaccaaggacctcaccaacggctgcgagatcaagccgtccgtcgccgccgcgccgcccgccgtgcagatcgccggcagggtcaacgagctcttcgctctggtcatgactgatccagatgctcctagccccagcgagccgactatgagagagtggcttcactggctggtggttaacataccaggtggaacagatccttctcaaggggatgtggtggtgccgtacatggggccacggccgccggtggggatccaccgctacgtgatggtgctgttccagcagaaggcgcgcgtggcggcgccgccgcccgacgaggacgccgcgcgcgccaggttcagcacgcgcgccttcgccgaccgccacgacctcggcctccccgtcgccgccctctacttcaacgcccagaaggagcccgccaaccgccgccgccgctactag

SEQ ID NO: 20 - protein, 176 aa, Oryza sativa
MASHVDPLVVGRVIGDVVDLFVPTTAMSVRFGTKDLTNGCEIKPSVAAAPPAVQIAGRVNELFALVMTDPDAPSPSEPTMREWLHWLVVNIPGGTDPSQGDVVVPYMGPRPPVGIHRYVMVLFQQKARVAAPPPDEDAARARFSTRAFADRHDLGLPVAALYFNAQKEPANRRRRY

SEQ ID NO: 21 - DNA, 516 bp, Sorghum bicolor
atggcgcggttcgtggatccgctggtggtggggcgggtgatcggcgaggtggtggacctgttcgtgccctccatctccatgaccgtcgcctatggccccaaggacatcagcaacggctgcctcctcaagccgtccgccaccgccgcgccgccgctcgtccgcatctccggccgccgcaacgacctctacacgctgatcatgacggaccctgatgcgcctagccccagcgacccgaccatgagggagtacctccactggatagtgaccaacataccaggaggaacggatgcaagcaaaggtgaggaggtggtggagtacatgggcccgcggccgccggtgggcatccaccgctacgtgctggtgctgttcgagcagaagacgcgcgtgcacgcggaggcgccccgcgagcgcgccaacttcaacacgcgcgcgttcgcggcggcgcacgagctcggcctccccaccgccgtcgtctacttcaacgcgcagaaggagcccgccaaccgccgccgctag

SEQ ID NO: 22 - protein, 171 aa, Sorghum bicolor
MARFVDPLVVGRVIGEVVDLFVPSISMTVAYGPKDISNGCLLKPSATAAPPLVRISGRRNDLYTLIMTDPDAPSPSDPTMREYLHWIVTNIPGGTDASKGEEVVEYMGPRPPVGIHRYVLVLFEQKTRVHAEAPRERANFNTRAFAAAHELGLPTAVVYFNAQKEPANRRR

SEQ ID NO: 23 - DNA, 20 bp, Artificial Sequence
  Guide RNA sequence
gaccactaggggatccacgg

SEQ ID NO: 24 - protein, 11 aa, Artificial Sequence
  MFT amino acid motif 1
VDPLVVGRVIG

SEQ ID NO: 25 - protein, 10 aa, Artificial Sequence
  MFT amino acid motif 2
MTDPDAPSPS

SEQ ID NO: 26 - protein, 13 aa, Artificial Sequence
  MFT amino acid motif 3
  VARIANT (4)..(4): Xaa is any amino acid
  VARIANT (9)..(9): Xaa is any amino acid
  VARIANT (10)..(10): Xaa is any amino acid
  VARIANT (11)..(11): Xaa is any amino acid
YFNXQKEPXXXRR

SEQ ID NO: 27 - protein, 16 aa, Artificial Sequence
  MFT amino acid motif 3A
  VARIANT (4)..(4): Xaa is S or A
  VARIANT (9)..(9): Xaa is A or V
  VARIANT (10)..(10): Xaa is V S or N
  VARIANT (11)..(11): Xaa is K or R
YFNXQKEPXXXRRREF

(19) United States
(12) Patent Application Publication
(10) Pub. No.: US 2023/0220409 A1
(43) Pub. Date: Jul. 13, 2023

(54) ALTERATION OF SEED COMPOSITION IN PLANTS

(71) Applicant: PIONEER HI-BRED INTERNATIONAL, INC., JOHNSTON, IA (US)

(72) Inventors: JANEL M BETTIS, URBANDALE, IA (US); JOHN D EVERARD, GRIMES, IA (US); KRISTIN HAUG COLLET, DES MOINES, IA (US); ZHAN-BIN LU, CLIVE, IA (US); BO SHEN, JOHNSTON, IA (US); SHREEDHARAN SRIRAM, WAUKEE, IA (US); YANG WANG, JOHNSTON, IA (US); GINA MARIE ZASTROW-HAYES, URBANDALE, IA (US)

(73) Assignee: PIONEER HI-BRED INTERNATIONAL, INC., JOHNSTON, IA (US)

(21) Appl. No.: 18/001,437

(22) PCT Filed: Jun. 2, 2021
(86) PCT No.: PCT/US21/35399

§ 371 Date: Dec. 9, 2022

Related U.S. Application Data
(60) Provisional application No. 63/038,312, filed on Jun. 12, 2020.

(51) Int. Cl. C12N 15/82; C07K 14/415; C12N 9/22; C12N 15/11

(52) U.S. Cl. CPC C12N 15/8247; C07K 14/415; C12N 15/8251; C12N 15/8213; C12N 9/22; C12N 15/11; C12N 2800/80; C12N 2310/20

(57) ABSTRACT

Provided are compositions comprising polynucleotides encoding modified MFT polypeptides. Also provided are recombinant DNA constructs, plants, plant cells, seed, and grain comprising the polynucleotides. Additionally, methods using the polynucleotides in plants to increase seed oil and/or protein content are also provided herein.

### REFERENCE TO SEQUENCE LISTING SUBMITTED ELECTRONICALLY

The official copy of the sequence listing is submitted electronically via EFS-Web as an ASCII formatted sequence listing with a file named 8467-US-PSP_SequenceListing_ST25.txt created on Jun. 3, 2020 and having a size of 34.2 kilobytes and is filed concurrently with the specification. The sequence listing comprised in this ASCII formatted document is part of the specification and is herein incorporated by reference in its entirety.

### FIELD

This disclosure relates to the field of molecular biology.

### BACKGROUND

Plant seeds are a source of useful products, such as protein and oil, for human and animal consumption. Thus, generating plants with seeds having increased protein or oil content may contribute to a higher-value crop. However, in many seeds oil content shows a strong negative correlation with seed protein content, as increasing seed protein content usually leads to a reduction of seed oil content. Further, it is difficult to break the negative correlation and increase both protein and oil content in the seed.

Therefore, there is a need to develop compositions and methods to generate plants that produce seeds with increased protein and/or oil content. This disclosure provides such compositions and methods.

### SUMMARY

Provided are modified polynucleotides encoding MFT polypeptides comprising an amino acid sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2, 10, 12, 14, 16, 18, 20, or 22, wherein the amino acid sequence comprises a non-leucine at the amino acid residue corresponding to position L140 of SEQ ID NO: 2. In certain embodiments the amino acid sequence comprises a glycine, asparagine, glutamine, alanine, serine, cysteine, or threonine at the amino acid residue corresponding to position L140 of SEQ ID NO: 2. In certain embodiments, the amino acid sequence comprises a serine at the amino acid residue corresponding to position L140 of SEQ ID NO: 2 (L1405).

Further provided are recombinant DNA constructs comprising any of the modified polynucleotides encoding the MFT polypeptides described herein. In certain embodiments, the recombinant DNA construct comprises a heterologous regulatory element (e.g., heterologous promoter) operably linked to the modified polynucleotide.

Also provided are plant cells comprising any of the modified polynucleotides encoding the MFT polypeptides described herein and plant cells comprising any of the recombinant DNA constructs described herein.

Further provided are plant cells comprising a polynucleotide encoding a MFT polypeptide comprising an amino acid sequence that is at least 80% identical to any one of SEQ ID NOs: 2, 10, 12, 14, 16, 18, 20, or 22 operably linked to a heterologous regulatory element.

Also provided are plant cells comprising decreased expression of a MFT polypeptide comprising an amino acid sequence that is at least 80% identical to any one of SEQ ID NOs: 2, 10, 12, 14, 16, 18, 20, or 22.

Further provided are plants and seeds that comprise the plant cell comprising a modified polynucleotide or a recombinant DNA construct described herein. In certain embodiments, the oil content of the seed is increased by a least at least about a 0.1%, 1.5%, 2%, 2.5%, 3%, 3.5%, 4%, 5%, 10%, or 15% and less than 20%, 15%, 10%, 9%, 8%, 7%, 6%, or 5% percentage point increase in total oil measured on a dry weight basis, or adjusted to 13% moisture, as compared to a control seed (e.g., seed comprising a non-modified polypeptide). In certain embodiments, the seed further comprises at 0.1%, 0.5%, 1%, 1.5%, 2%, 2.5%, 3%, 3.5%, 4%, 4.5%, or 5% and less than 15%, 14%, 13%, 12%, 11%, 10%, 9%, 8%, 7%, 6%, 5%, 4.5%, 4%, 3.5%, 3%, 2.5%, 2%, 1.5%, 1%, or 0.5% percentage point increase in total protein measured on a dry weight basis, or adjusted to 13% moisture, as compared to a control seed (e.g., seed comprising a non-modified polypeptide).

Also provided is a method of producing a plant producing seeds having increased oil and/or protein content comprising expressing in a plant any of the modified polynucleotides described herein. In certain embodiments, the method comprises expressing in a regenerable plant cell any of the recombinant DNA constructs comprising the modified polynucleotides described herein and generating a plant from the plant cell, wherein the plant comprises the modified polynucleotide and produces seeds having an increased oil content as compared to a control plant not comprising the polynucleotide. In certain embodiments, the method comprises introducing into a regenerable plant cell a targeted genetic modification of an endogenous gene encoding an MFT protein to produce any of the modified MFT polynucleotides described herein and generating a plant from the plant cell, wherein the plant comprises the polynucleotide and produces seeds having an increased oil content as compared to a control plant not comprising the polynucleotide. In certain embodiments, the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), and engineered site-specific meganucleases, or Argonaute.

Further provided is a method of producing a seed having increased oil content comprising crossing a first plant line comprising a polynucleotide encoding a polypeptide that is at least 80% identical to any one of SEQ ID NOs: 2, 10, 12, 14, 16, 18, 20, or 22, the polypeptide comprising a modification at a position other than the amino acid corresponding to L106 in SEQ ID NO: 2 that increases oil content in the first plant with a second different plant line and harvesting the seed produced thereby.

### BRIEF DESCRIPTION OF THE DRAWINGS AND THE SEQUENCE LISTING

The disclosure can be more fully understood from the following detailed description and the accompanying drawings and Sequence Listing, which form a part of this application.

FIG. 1 provides a sequence alignment of the MFT polynucleotide sequences of a wild-type MFT (SEQ ID NO: 1), the HiPO #358 MFT sequence (SEQ ID NO: 3), and the MFT sequence from Glycine soja (SEQ ID NO: 5).

FIG. 2 provides a sequence alignment of the MFT amino acid sequences of a wild-type MFT (SEQ ID NO: 2), the HiPO #358 MFT sequence (SEQ ID NO: 4), and the MFT sequence from Glycine soja (SEQ ID NO: 6).

FIGS. 3A-3D provides a sequence alignment of the MFT allele from a Glycine max variety (SEQ ID NO: 7) and Glycine soja (SEQ ID NO: 8).

FIG. 4 provides an amino acid sequence alignment of MFT from Glycine max wild-type (SEQ ID NO: 2), Glycine max HiPO #358 (SEQ ID NO: 4), Glycine soja (SEQ ID NO: 6), Brassica napus (SEQ ID NO: 10), Gossypium raimondii (SEQ ID NO: 12), Zea mays (SEQ ID NO: 14), Triticum asestivum (SEQ ID NO: 16), Medicago truncatula (SEQ ID NO: 18), Oryza sativa (SEQ ID NO: 20), and Sorghum bicolor (SEQ ID NO: 22). Three highly conserved domains are underlined.

The sequence descriptions (Table 1) summarize the Sequence Listing attached hereto, which is hereby incorporated by reference. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).

TABLE 1
Sequence Listing Description

| SEQ ID NO: | Name | Organism |
|---|---|---|
| 1 | MFT coding sequence | Glycine max |
| 2 | MFT amino acid sequence | Glycine max |
| 3 | MFT coding sequence HiPO# | Glycine max |
| | 538 mutant | |
| 4 | MFT amino acid sequence | Glycine max |
| | HiPO# 538 mutant | |
| 5 | MFT coding sequence | Glycine soja |
| 6 | MFT amino acid sequence | Glycine soja |
| 7 | MFT genomic sequence | Glycine max |
| 8 | MFT genomic sequence | Glycine soja |
| 9 | MFT coding sequence | Brassica napus |
| 10 | MFT amino acid sequence | Brassica napus |
| 11 | MFT coding sequence | Gossypium raimondii |
| 12 | MFT amino acid sequence | Gossypium raimondii |
| 13 | MFT coding sequence | Zea mays |
| 14 | MFT amino acid sequence | Zea mays |
| 15 | MFT coding sequence | Triticum asestivum |
| 16 | MFT amino acid sequence | Triticum asestivum |
| 17 | MFT coding sequence | Medicago truncatula |
| 18 | MFT amino acid sequence | Medicago truncatula |
| 19 | MFT coding sequence | Oryza sativa |
| 20 | MFT amino acid sequence | Oryza sativa |
| 21 | MFT coding sequence | Sorghum bicolor |
| 22 | MFT amino acid sequence | Sorghum bicolor |
| 23 | MFT-CR1 | |
| 24 | MFT amino acid motif 1 | |
| 25 | MFT amino acid motif 2 | |
| 26 | MFT amino acid motif 3 | |
| 27 | MFT amino acid motif 3A | |

### DETAILED DESCRIPTION

### I. Compositions

### A. MFT Polynucleotide and Polypeptides

The present disclosure provides polynucleotides encoding Mother of FT (flowering time) and TFL1 (terminated flowering locus1) (referred to herein as MFT) polypeptides, which are members of the phosphatidylethanolamine binding protein (PEBP) family.

One aspect of the disclosure provides a polynucleotide encoding an MFT polypeptide comprising an amino acid sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2, 10, 12, 14, 16, 18, 20, or 22, wherein the amino acid sequence comprises a non-leucine (e.g., alanine, arginine, asparagine, aspartic acid, cysteine, glutamine, glutamic acid, glycine, histidine, isoleucine, lysine, methionine, phenylalanine, proline, serine, threonine, tryptophan, tyrosine, and valine) at the amino acid residue corresponding to position L140 of SEQ ID NO: 2. In certain embodiments, the non-leucine at the residue corresponding to position L140 of SEQ ID NO: 2 is introduced by a substitution mutation. In certain embodiments, the non-leucine at the residue corresponding to position L140 of SEQ ID NO: 2 is introduced by a deletion mutation, such as, for example, a deletion of L140. In certain embodiments, the non-leucine at the residue corresponding to position L140 of SEQ ID NO: 2 is introduced by an insertion mutation, such as, for example, the insertion of any amino acid, other than leucine, at position 140.

In certain embodiments the mutation is a substitution of a glycine, asparagine, glutamine, alanine, serine, cysteine, or threonine at the amino acid residue corresponding to position L140 of SEQ ID NO: 2. In certain embodiments, the MFT polypeptide comprises a leucine to serine substitution at the amino acid residue corresponding to L140 (L1405) of SEQ ID NO: 2.

In certain embodiments, the MFT polypeptide further comprises a leucine at the amino acid residue corresponding to position L106 of SEQ ID NO: 2.

In certain embodiments, the MFT polypeptides described herein comprise at least one amino acid motif selected from the group consisting of VDPLVVGRVIG (SEQ ID NO: 24), MTDPDAPSPS (SEQ ID NO: 25), and YFNX₁QKEPX₂X₃X₄RR (SEQ ID NO: 26), where X is any amino acid. In certain embodiments, the MFT polypeptides described herein comprise each of the amino acid motifs VDPLVVGRVIG (SEQ ID NO: 24), MTDPDAPSPS (SEQ ID NO: 25), and YFNX₁QKEPX₂X₃X₄RR (SEQ ID NO: 26), where X is any amino acid. In certain embodiments, X₁ is S or A, X₂ is A or V, X₃ is V, S, or N, and X₄ is K or R. In certain embodiments, the amino acid motif VDPLVVGRVIG (SEQ ID NO: 24) is present from amino acid positions 23 to 33 corresponding to SEQ ID NO: 2. In certain embodiments, the amino acid motif MTDPDAPSPS (SEQ ID NO: 25) is present from amino acid positions 85 to 94 corresponding to SEQ ID NO: 2. In certain embodiments, the amino acid motif YFNX₁QKEPX₂X₃X₄RR (SEQ ID NO: 26) is present from amino acid positions 178 to 190 corresponding to SEQ ID NO: 2.

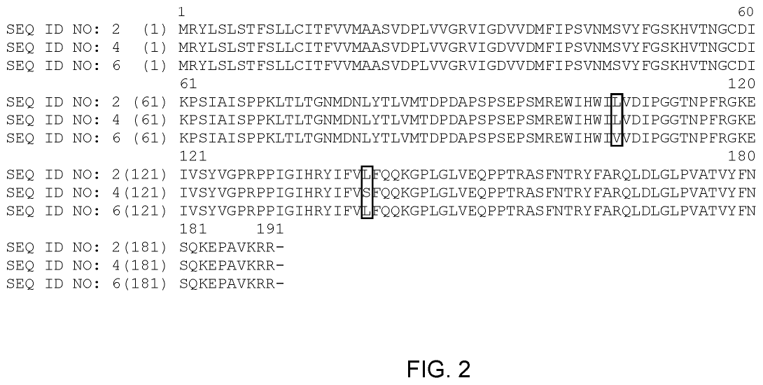

FIG. 2 provides a sequence alignment that shows the amino acid residues in SEQ ID NOs: 10, 12, 14, 16, 18, 20, and 22 that correspond to residues L140, L106, positions 23 to 33, positions 85 to 94, and positions 178 to 190 of SEQ ID NO: 1.

As used herein an “amino acid deletion,” “deletion mutation,” or the like, refers to a mutation in which the indicated amino acid residue is removed from the polypeptide sequence, so that, when aligned to the reference sequence (e.g., SEQ ID NO: 2) the mutated sequence does not have an amino acid corresponding to the indicated position of the reference sequence. An “amino acid addition,” “addition mutation,” “amino acid insertion,” “insertion,” or the like, refers to a mutation in which at least one amino acid residue is added to the polypeptide sequence, so that, when aligned to the reference sequence (e.g., SEQ ID NO: 2) the mutated sequence contains an additional amino acid corresponding to the indicated position of the reference sequence.

An “amino acid substitution,” “substitution mutation,” or the like, refers to a mutation in which the indicated amino acid residue is replaced with a different amino acid residue, so that, when aligned to the reference sequence (e.g., SEQ ID NO: 2) the mutated sequence does not have the same amino acid at the indicated position. When the amino acid residue is substituted for a residue that has similar properties (e.g., size, charge, and/or hydrophobicity) the substitution is referred to as a conservative amino substitution. Conservative amino acid substitutions are well known in the art. Alternatively, when the amino acid residue is substituted for an amino acid that has dissimilar properties the mutation is referred to as a radical amino acid substitution.

As used herein, a “mutation” refers a polynucleotide or polypeptide that has been altered through human intervention. Such that a “mutated polynucleotide” or “mutated polypeptide” has a sequence that differs from the sequence of the corresponding non-mutated polynucleotide or polypeptide by at least one nucleotide or amino acid. In certain embodiments of the disclosure, the mutated polynucleotide or polynucleotide comprises an alteration that results from a guide polynucleotide/Cas endonuclease system as disclosed herein. A mutated or modified plant is a plant comprising a mutated polynucleotide or polypeptide.

In certain embodiments, the MFT polypeptides encoded by the modified polynucleotides described herein (e.g., MFT polypeptide comprising a non-leucine at the amino acid residue corresponding to position L140 of SEQ ID NO: 2) have an increase in activity as compared to control polypeptide not comprising the modification. In certain embodiments, the modified polypeptide comprises at least a 1%, 5%, 10%, 25%, 50%, 100%, 200%, 400%, 500%, 1000% and less than a 10,000%, 5000%, 2500%, 1000%, 900%, 800%, 700%, 600%, 500%, 400%, 300%, 200% or 100% increase in activity as compared to the control polypeptide. As used herein, “increase in activity” “increased activity” and the like refers to any detectable gain in activity of the polypeptide. The increase in activity can be any MFT activity known in the art.

As used herein “encoding,” “encoded,” or the like, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference).

The terms “polypeptide,” “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

As used herein “percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Unless otherwise stated, sequence identity/similarity values provided herein refer to the value obtained using the BLAST 2.0 suite of programs using default parameters (Altschul, et al., (1997) Nucleic Acids Res. 25:3389-402).

### B. Recombinant DNA Construct

Also provided is a recombinant DNA construct comprising any of the MFT polynucleotides described herein. In certain embodiments, the recombinant DNA construct further comprises at least one regulatory element. In certain embodiments, the at least one regulatory element of the recombinant DNA construct comprises a promoter. In certain embodiments, the promoter is heterologous to the MFT polynucleotide sequence.

As used herein, a “recombinant DNA construct” comprises two or more operably linked DNA segments, preferably DNA segments that are not operably linked in nature (i.e., heterologous). Non-limiting examples of recombinant DNA constructs include a polynucleotide of interest operably linked to regulatory elements, which aid in the expression, autologous replication, and/or genomic insertion of the sequence of interest. Such regulatory elements include, for example, promoters, expression modulating elements (EMEs), termination sequences, enhancers, etc., or any component of an expression cassette; a plasmid, cosmid, virus, autonomously replicating sequence, phage, or linear or circular single-stranded or double-stranded DNA or RNA nucleotide sequence; and/or sequences that encode heterologous polypeptides.

The MFT polynucleotides described herein can be provided in expression cassettes for expression in a plant of interest or any organism of interest. The cassette can include 5′ and 3′ regulatory sequences operably linked to a MFT polynucleotide. “Operably linked” is intended to mean a functional linkage between two or more elements. For, example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the MFT polynucleotide to be under the transcriptional regulation of the regulatory regions. The expression cassette may additionally contain selectable marker genes.

The expression cassette can include in the 5′-3′ direction of transcription, a transcriptional and translational initiation region (e.g., a promoter), a MFT polynucleotide, and a transcriptional and translational termination region (e.g., termination region) functional in plants. The regulatory regions (e.g., promoters, transcriptional regulatory regions, and translational termination regions) and/or the MFT polynucleotide may be native/analogous to the host cell or to each other. Alternatively, the regulatory regions and/or the MFT polynucleotide may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide that is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region, with the plant host, or may be derived from another source (i.e., foreign or heterologous) than the promoter, the MFT polynucleotide, the plant host, or any combination thereof.

The expression cassette may additionally contain a 5′ leader sequences. Such leader sequences can act to enhance translation. Translation leaders are known in the art and include viral translational leader sequences.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

As used herein “promoter” refers to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Certain types of promoters preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026); GOS2 (U.S. Pat. No. 6,504,083), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Also contemplated are synthetic promoters which include a combination of one or more heterologous regulatory elements.

The promoter of the recombinant DNA constructs described herein can be any type or class of promoter known in the art, such that any one of a number of promoters can be used to express the various MFT polynucleotide sequences disclosed herein, including the native promoter of the polynucleotide sequence of interest. The promoters for use in the recombinant DNA constructs of the invention can be selected based on the desired outcome.

In certain embodiments, the recombinant DNA construct, described herein, is expressed in a plant or seed. In certain embodiments, the plant or seed is a soybean plant or soybean seed. The polynucleotides or recombinant DNA constructs disclosed herein may be used for transformation of any plant species

### C. Plants and Plant Cells

Provided are plants, plant cells, plant parts, seeds, and grain comprising at least one of the MFT polynucleotide sequences or recombinant DNA constructs, described herein, so that the plants, plant cells, plant parts, seeds, and/or grain express any of the MFT polypeptides described herein. In certain embodiments, the plants, plant cells, plant parts, seeds, and/or grain have stably incorporated at least one MFT polynucleotide into its genome. In certain embodiments, the plants, plant cells, plant parts, seeds, and/or grain can comprise multiple MFT polynucleotides (i.e., at least 1, 2, 3, 4, 5, 6 or more).

Also provided are plants, plant cells, plant parts, seeds, and grain comprising a polynucleotide encoding a MFT polypeptide comprising an amino acid sequence that is at least 80% identical to any one of SEQ ID NOs: 2, 10, 12, 14, 16, 18, 20, or 22 operably linked to a heterologous regulatory element. In certain embodiments, the heterologous regulatory element is a heterologous promoter.

Further provided are plants, plant cells, plant parts, seeds, and grain comprising a polynucleotide encoding a MFT polypeptide comprising an amino acid sequence that is at least 80% identical to any one of SEQ ID NOs: 2, 10, 12, 14, 16, 18, 20, or 22.

In certain embodiments, the seeds and plant have an increase in total oil content when compared to a seed or plant comprising a comparable polynucleotide which lacks the modification.

In certain embodiments, the oil content in the seed containing or expressing the modified polynucleotides or polypeptides disclosed herein comprises an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the oil content measured on a dry weight basis, or adjusted to 13% moisture, of a control seed (e.g., seed expressing the polypeptide without the modifications). In certain embodiments, the oil content in the seed containing or expressing the modified polynucleotides or polypeptides disclosed herein comprises at least about a 0.1%, 1.5%, 2%, 2.5%, 3%, 3.5%, 4%, 5%, 10%, or 15% and less than 20%, 15%, 10%, 9%, 8%, 7%, 6%, or 5% percentage point increase in total oil measured on a dry weight basis, or adjusted to 13% moisture, as compared to a control seed (e.g., seed comprising a non-modified polypeptide).

In certain embodiments, the seeds and plant have an increase in total protein content when compared to a seed or plant comprising a comparable polynucleotide which lacks the modification.

In certain embodiments, the protein content in the seed containing or expressing the modified polynucleotides or polypeptides disclosed herein comprises an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the protein content measured on a dry weight basis, or adjusted to 13% moisture, of a control seed (e.g., seed expressing the polypeptide without the modifications). In certain embodiments, the protein content in the seed containing or expressing the modified polynucleotides or polypeptides disclosed herein comprises at least about a 0.1%, 0.5%, 1%, 1.5%, 2%, 2.5%, 3%, 3.5%, 4%, 4.5%, or 5% and less than 15%, 14%, 13%, 12%, 11%, 10%, 9%, 8%, 7%, 6%, 5%, 4.5%, 4%, 3.5%, 3%, 2.5%, 2%, 1.5%, 1%, or 0.5% percentage point increase in total protein measured on a dry weight basis, or adjusted to 13% moisture, as compared to a control seed (e.g., seed comprising a non-modified polypeptide).

In certain embodiments, the seeds and plant have an increase in both total protein and total oil content when compared to a control seed or plant (e.g., a seed or plant comprising a comparable polynucleotide which lacks the modification). The increase in total oil content and total protein content can be any increase described herein.

In certain embodiments, the seeds and plant have modified amounts of fatty acids when compared to a control seed or plant, such as a seed or plant comprising a comparable polynucleotide which lacks the modification.

In certain embodiments, the linoleic acid content in the seed containing or expressing the modified polynucleotides or polypeptides disclosed herein comprises an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the linoleic acid content of a control seed (e.g., seed expressing the polypeptide without the modifications). In certain embodiments, the linoleic acid content in the seed containing or expressing the modified polynucleotides or polypeptides disclosed herein comprises at least about a 0.1%, 0.5%, 1%, 1.5%, 2%, 2.5%, 3%, 3.5%, 4%, 4.5%, or 5% and less than 15%, 14%, 13%, 12%, 11%, 10%, 9%, 8%, 7%, 6%, 5%, 4.5%, 4%, 3.5%, 3%, 2.5%, 2%, 1.5%, 1%, or 0.5% percentage point increase in linoleic acid content as compared to a control seed.

In certain embodiments, the linolenic acid content in the seed containing or expressing the modified polynucleotides or polypeptides disclosed herein comprises an decrease of at least 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the linolenic acid content of a control seed (e.g., seed expressing the polypeptide without the modifications). In certain embodiments, the linolenic acid content in the seed containing or expressing the modified polynucleotides or polypeptides disclosed herein comprises at least about a −4%, −3.5%, −3%, −2.5%, −2%, −1.5%, −1%, −0.5%, 0%, 0.5%, 1%, 1.5%, 2%, 2.5%, 3%, 3.5%, 4%, 4.5%, or 5% and less than 15%, 14%, 13%, 12%, 11%, 10%, 9%, 8%, 7%, 6%, 5%, 4.5%, 4%, 3.5%, 3%, 2.5%, 2%, 1.5%, 1%, or 0.5% percentage point change in linolenic acid content as compared to a control seed.

In certain embodiments, the plants comprising the modified polynucleotide encoding the MFT polypeptide have a yield that is greater than or within 0.5%, 1%, 1.5%, 2%, 2.5%, 3%, 3.5%, 4%, 4.5%, or 5%, as compared to the corresponding control plant, for example, one which has a similar genetic background but lacks the introduced mutations.

As used herein, “yield” refers to the amount of agricultural production harvested per unit of land and may include reference to bushels per acre or kilograms per hectare of a crop at harvest, as adjusted for grain moisture. Grain moisture is measured in the grain at harvest. The adjusted test weight of grain is determined to be the weight in pounds per bushel or kilogram, adjusted for grain moisture level at harvest.

In certain embodiments, the plants described herein are elite plant lines (e.g., elite soybean line). In certain embodiments, the plant cells, plant parts, seeds, and grain are isolated from or produced by an elite plant line. As used herein, “elite line” refers to any line that has resulted from breeding and selection for superior agronomic performance that allows a producer to harvest a product of commercial significance. Numerous elite lines are available and known to those of skill in the art of plant breeding (e.g., soybean, canola, and sunflower breeding). An “elite population” is an assortment of elite individuals or lines that can be used to represent the state of the art in terms of agronomically superior genotypes of a given crop species, such as soybean.

In certain embodiments, the modified MFT polynucleotide is operably linked to a heterologous regulatory element, such as but not limited to a constitutive, tissue-preferred, or other promoter for expression in plants or a constitutive enhancer.

In certain embodiments, the modified MFT polynucleotide described herein is introduced into the plants, plant cells, plant parts, seeds, and grain by a targeted genetic modification at a genomic locus that encodes an endogenous MFT polypeptide, such that the plant, plant cell, plant part, seed, or grain encodes any of the MFT polypeptides described herein, for example, a MFT polypeptide comprising an amino acid sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 2, 10, 12, 14, 16, 18, 20, or 22, wherein the amino acid sequence comprises a non-leucine at the amino acid residue corresponding to position L140 of SEQ ID NO: 2.

In certain embodiments, the genomic locus that encodes an endogenous MFT polypeptide comprises an polynucleotide sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 7. The MFT genomic locus of SEQ ID NO: 7 comprises a promoter corresponding to nucleotides 1-1431, a 5-UTR corresponding to nucleotides 1432-1469, exons corresponding to nucleotides 1470-1718, 1813-1874, 1967-2007, and 3001-3221, introns corresponding to nucleotides 1719-1812, 1875-1966, and 2008-3000, and a 3′-UTR corresponding to nucleotides 3222-3468 of SEQ ID NO: 7.

A “genomic locus” as used herein, generally refers to the location on a chromosome of the plant where a gene, such as a polynucleotide encoding a MFT polypeptide, is found. As used herein, “gene” includes a nucleic acid fragment that expresses a functional molecule such as, but not limited to, a specific protein coding sequence and regulatory elements, such as those preceding (5′ non-coding sequences) and following (3′ non-coding sequences) the coding sequence.

A “regulatory element” generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e., it activates expression of genes located on the same nucleic acid molecule, e.g., a chromosome, where the regulatory element is located.

An “enhancer” element is any nucleic acid molecule that increases transcription of a nucleic acid molecule when functionally linked to a promoter regardless of its relative position. A “repressor” (also sometimes called herein silencer) is defined as any nucleic acid molecule which inhibits the transcription when functionally linked to a promoter regardless of relative position. The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription. An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature mRNA derived from the gene but is not necessarily a part of the sequence that encodes the final gene product. The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes. The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“Genetic modification,” “DNA modification,” and the like refers to a site-specific modification that alters or changes the nucleotide sequence at a specific genomic locus of the plant. The genetic modification of the compositions and methods described herein may be any modification known in the art such as, for example, insertion, deletion, single nucleotide polymorphism (SNP), and or a polynucleotide modification. Additionally, the targeted DNA modification in the genomic locus may be located anywhere in the genomic locus, such as, for example, a coding region of the encoded polypeptide (e.g., exon), a non-coding region (e.g., intron), a regulatory element, or untranslated region.

As used herein, a “targeted” genetic modification or “targeted” DNA modification, refers to the direct manipulation of an organism's genes. The targeted modification may be introduced using any technique known in the art, such as, for example, plant breeding, genome editing, or single locus conversion.

The DNA modification of the genomic locus may be done using any genome modification technique known in the art or described herein. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In certain embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

As used herein, the term “plant” includes plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides.

The polynucleotides or recombinant DNA constructs disclosed herein may be used for transformation of any plant species, including, but not limited to, monocots and dicots. Additionally, the genetic modifications described herein may be used to modify any plant species, including, but not limited to, monocots and dicots.

Examples of plant species of interest include, but are not limited to, maize (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), peanuts (Arachis hypogaea), cotton (Gossypium barbadense, Gossypium hirsutum), coconut (Cocos nucifera), olive (Olea europaea), cashew (Anacardium occidentale), macadamia (Macadamia integrifolia), almond (Prunus amygdalus), green beans (Phaseolus vulgaris), lima beans (Phaseolus limensis), and peas (Lathyrus spp.).

In certain embodiments, plants of the present disclosure are oil-seeds plants such as, but not limited to, cotton, soybean, safflower, sunflower, Brassica, maize, alfalfa, palm, and coconut. In certain embodiments, soybean, sunflower, and/or Brassica plants are optimal, and in yet other embodiments soybean plants are optimal.

For example, in certain embodiments, soybean plants are provided that comprise, in their genome, a polynucleotide that encodes an MFT polypeptide comprising an amino acid sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2, 10, 12, 14, 16, 18, 20, or 22, wherein the amino acid sequence comprises a non-leucine at the amino acid residue corresponding to position L140 of SEQ ID NO: 2.

### D. Stacking Other Traits of Interest

In some embodiments, the MFT polynucleotides disclosed herein are engineered into a molecular stack. Thus, the various host cells, plants, plant cells, plant parts, seeds, and/or grain disclosed herein can further comprise one or more traits of interest. In certain embodiments, the host cell, plant, plant part, plant cell, seed, and/or grain is stacked with any combination of polynucleotide sequences of interest in order to create plants with a desired combination of traits. As used herein, the term “stacked” refers to having multiple traits present in the same plant or organism of interest. For example, “stacked traits” may comprise a molecular stack where the sequences are physically adjacent to each other. A trait, as used herein, refers to the phenotype derived from a particular sequence or groups of sequences. In one embodiment, the molecular stack comprises at least one polynucleotide that confers tolerance to glyphosate. Polynucleotides that confer glyphosate tolerance are known in the art.

In certain embodiments, the molecular stack comprises at least one polynucleotide that confers tolerance to glyphosate and at least one additional polynucleotide that confers tolerance to a second herbicide.

In certain embodiments, the plant, plant cell, seed, and/or grain having an inventive polynucleotide sequence may be stacked with, for example, one or more sequences that confer tolerance to: an ALS inhibitor; an HPPD inhibitor; 2,4-D; other phenoxy auxin herbicides; aryloxyphenoxypropionate herbicides; dicamba; glufosinate herbicides; herbicides which target the protox enzyme (also referred to as “protox inhibitors”).

The plant, plant cell, plant part, seed, and/or grain comprising a polynucleotide sequence disclosed herein can also be combined with at least one other trait to produce plants that further comprise a variety of desired trait combinations. For instance, the plant, plant cell, plant part, seed, and/or grain having the polynucleotide sequence may be stacked with polynucleotides encoding polypeptides having pesticidal and/or insecticidal activity, or a plant, plant cell, plant part, seed, and/or grain comprising a polynucleotide sequence provided herein may be combined with a plant disease resistance gene.

In certain embodiments, the molecular stack comprises at least one additional polynucleotide that confers increased seed protein or oil content. For instance, a modified polynucleotide encoding a diacylglycerol acyltransferase (DGAT) polypeptide, such as those described in WO19/232182, or a high oleic acid trait, such as those described in U.S. Pat. No. 8,609,935.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

Any plant having an inventive polynucleotide sequence disclosed herein can be used to make a food or a feed product. Such methods comprise obtaining a plant, explant, seed, plant cell, or cell comprising the polynucleotide sequence and processing the plant, explant, seed, plant cell, or cell to produce a food or feed product.

### II. Methods

A. Methods for Increasing Seed Oil and/or Protein Content

Provided are methods for increasing seed oil and/or protein content comprising expressing in a plant a modified polynucleotide encoding any of the MFT polypeptides described herein.

In certain embodiments, the method comprises: expressing in a regenerable plant cell a recombinant DNA construct comprising a polynucleotide described herein; and generating the plant from the plant cell. In certain embodiments, the polynucleotide is operably linked to at least one regulatory sequence. In certain embodiments, the at least one regulatory sequence is a heterologous promoter. The recombinant DNA construct for use in the method may be any recombinant DNA construct provided herein. In certain embodiments the recombinant DNA is expressed by introducing into a plant, plant cell, plant part, seed, and/or grain the recombinant DNA construct, whereby the polypeptide is expressed in the plant, plant cell, plant part, seed, and/or grain. In certain embodiments the recombinant DNA construct is incorporated into the genome of the plant.

Various methods can be used to introduce the MFT sequences (e.g., modified MFT sequence or recombinant DNA comprising the modified MFT sequence) into a plant, plant part, plant cell, seed, and/or grain. “Introducing” is intended to mean presenting to the plant, plant cell, seed, and/or grain the inventive polynucleotide or resulting polypeptide in such a manner that the sequence gains access to the interior of a cell of the plant. The methods of the disclosure do not depend on a particular method for introducing a sequence into a plant, plant cell, seed, and/or grain, only that the polynucleotide or polypeptide gains access to the interior of at least one cell of the plant.

“Stable transformation” is intended to mean that the polynucleotide introduced into a plant integrates into the genome of the plant of interest and is capable of being inherited by the progeny thereof “Transient transformation” is intended to mean that a polynucleotide is introduced into the plant of interest and does not integrate into the genome of the plant or organism or a polypeptide is introduced into a plant or organism.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606), Agrobacterium-mediated transformation (U.S. Pat. Nos. 5,563,055 and 5,981,840), Ochrobacterium-mediated transformation (U.S. Patent Application Publication 2018/0216123 and WO20/092494) direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the MFT sequences can be provided to a plant using a variety of transient transformation methods. Such transient transformation methods include, but are not limited to, the introduction of the MFT protein directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference.

In other embodiments, the inventive polynucleotides disclosed herein may be introduced into plants by contacting plants with a virus or viral nucleic acids. Generally, such methods involve incorporating a nucleotide construct of the disclosure within a DNA or RNA molecule. It is recognized that the inventive polynucleotide sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide disclosed herein can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided, and the transfer cassette is integrated at the target site. The polynucleotide of interest is thereby integrated at a specific chromosomal position in the plant genome. Other methods to target polynucleotides are set forth in WO 2009/114321 (herein incorporated by reference), which describes “custom” meganucleases produced to modify plant genomes, in particular the genome of maize. See, also, Gao et al. (2010) Plant Journal 1:176-187.

One of skill will recognize that after the expression cassette containing the inventive polynucleotide is stably incorporated in transgenic plants and confirmed to be operable, it can be introduced into other plants by sexual crossing. Any of a number of standard breeding techniques can be used, depending upon the species to be crossed.

Parts obtained from the regenerated plants described herein, such as flowers, seeds, leaves, branches, fruit, and the like are included, provided that these parts comprise cells comprising the inventive polynucleotide. Progeny and variants, and mutants of the regenerated plants are also included, provided that these parts comprise the introduced nucleic acid sequences.

In one embodiment, a homozygous transgenic plant can be obtained by sexually mating (selfing) a heterozygous transgenic plant that contains a single added heterologous nucleic acid, germinating some of the seed produced and analyzing the resulting plants produced for altered cell division relative to a control plant (i.e., native, non-transgenic). Back-crossing to a parental plant and out-crossing with a non-transgenic plant are also contemplated.

In certain embodiments, the method comprises: modifying an endogenous MFT gene in a plant to encode any of the MFT polypeptides described herein. In certain embodiments, the method comprises introducing into a regenerable plant cell a targeted genetic modification of an endogenous MFT gene to produce any of the modified MFT polypeptides described herein and generating a plant from the plant cell.

In certain embodiments, the method comprises providing a guide RNA, at least one polynucleotide modification template, and at least one Cas endonuclease to a plant cell, wherein the at least one Cas endonuclease introduces a double stranded break at an endogenous MFT gene in the plant cell and generates any of the modified polynucleotides described herein, obtaining a plant from the plant cell; and generating a progeny plant that comprises the polynucleotide and produces seeds having an increased oil content as compared to a control plant not comprising the polynucleotide

Various methods can be used to introduce a genetic modification at a genomic locus that encodes an MFT polypeptide into the plant, plant part, plant cell, seed, and/or grain. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In certain embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

TAL effector nucleases (TALEN) are a class of sequence-specific nucleases that can be used to make double-strand breaks at specific target sequences in the genome of a plant or other organism (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

Genome editing using DSB-inducing agents, such as Cas9-gRNA complexes, has been described, for example in U.S. Patent Application US 2015-0082478 A1, WO2015/026886 A1, WO2016007347, and WO201625131 all of which are incorporated by reference herein.

In certain embodiments the genetic modification is introduced without introducing a double strand break using base editing technology, see e.g., Gaudelli et al., (2017) Programmable base editing of A*T to G*C in genomic DNA without DNA cleavage. Nature 551(7681):464-471; Komor et al., (2016) Programmable editing of a target base in genomic DNA without double-stranded DNA cleavage, Nature 533(7603):420-4.

In certain embodiments, base editing comprises (i) a catalytically impaired CRISPR-Cas9 mutant that is mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; or (iv) nickase activity to cleave the non-edited DNA strand, followed by cellular DNA repair processes to replace the G-containing DNA strand.

In certain embodiments, the plant generated from the methods described herein produce seeds that have an increase in total oil content when compared to a seed or plant comprising a comparable polynucleotide which lacks the modification.

In certain embodiments, the oil content in the seeds of the plants produced by the methods described herein comprise an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the oil content measured on a dry weight basis, or adjusted to 13% moisture, of a control seed (e.g., seed expressing the polypeptide without the modifications). In certain embodiments, the oil content in the seeds of the plants produced by the methods described herein comprise at least about a 0.1%, 1.5%, 2%, 2.5%, 3%, 3.5%, 4%, 5%, 10%, or 15% and less than 20%, 15%, 10%, 9%, 8%, 7%, 6%, or 5% percentage point increase in total oil measured on a dry weight basis, or adjusted to 13% moisture, as compared to a control seed (e.g., seed comprising a non-modified polypeptide).

In certain embodiments, the plant generated from the methods described herein produce seeds having an increase in total protein content when compared to a seed or plant comprising a comparable polynucleotide which lacks the modification.

In certain embodiments, the protein content in the seeds of the plants produced by the methods described herein comprise an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the protein content measured on a dry weight basis, or adjusted to 13% moisture, of a control seed (e.g., seed expressing the polypeptide without the modifications). In certain embodiments, the protein content in the seeds of the plants produced by the methods described herein comprise at least about a 0.1%, 0.5%, 1%, 1.5%, 2%, 2.5%, 3%, 3.5%, 4%, 4.5%, or 5% and less than 15%, 14%, 13%, 12%, 11%, 10%, 9%, 8%, 7%, 6%, 5%, 4.5%, 4%, 3.5%, 3%, 2.5%, 2%, 1.5%, 1%, or 0.5% percentage point change in total protein measured on a dry weight basis, or adjusted to 13% moisture, as compared to a control seed (e.g., seed comprising a non-modified polypeptide).

In certain embodiments, the plants generated from the methods described herein produce seeds having an increase in both total protein and total oil content when compared to a seed or plant comprising a comparable polynucleotide which lacks the modification. The increase in total oil content and total protein content can be any increase described herein.

In certain embodiments, the plants generated from the methods described have a yield that is greater than or within 0.5%, 1%, 1.5%, 2%, 2.5%, 3%, 3.5%, 4%, 4.5%, or 5%, as compared to the corresponding control plant, for example, one which has a similar genetic background but lacks the introduced mutations.

B. Methods for Modifying Seed Oil and/or Protein Content

Also provided are methods for modifying seed oil and/or protein content comprising modulating the expression of a polynucleotide encoding a MFT polypeptide comprising an amino acid sequence that is at least 80% identical to any one of SEQ ID NOs: 2, 10, 12, 14, 16, 18, 20, or 22. In certain embodiments, the method generate plants producing seeds having increased seed oil and/or seed protein content. The increase in seed oil and/or protein may be any increase described herein. In certain embodiments, the plants have a yield that is greater than or within 0.5%, 1%, 1.5%, 2%, 2.5%, 3%, 3.5%, 4%, 4.5%, or 5%, as compared to the corresponding control plant.

In certain embodiments, the method comprises introducing into a regenerable plant cell a recombinant DNA construct comprising a polynucleotide encoding a MFT polypeptide comprising an amino acid sequence that is at least 80% identical to any one of SEQ ID NOs: 2, 10, 12, 14, 16, 18, 20, or 22 and generating the plant, wherein the level or activity of the encoded polypeptide is increased in the plant compared to a control plant.

In certain embodiments, the method comprises introducing in a regenerable plant cell a targeted genetic modification at a genomic locus that encodes a MFT polypeptide comprising an amino acid sequence this is at least 80% identical to any one of SEQ ID NOs: 2, 10, 12, 14, 16, 18, 20, or 22 and generating the plant, wherein the level or activity of the encoded polypeptide is increased in the plant compared to a control plant.

In certain embodiments, the method comprises introducing in a regenerable plant cell a targeted genetic modification at a genomic locus that encodes a MFT polypeptide comprising an amino acid sequence this is at least 80% identical to any one of SEQ ID NOs: 2, 10, 12, 14, 16, 18, 20, or 22 and generating the plant, wherein the level or activity of the encoded polypeptide is decreased in the plant compared to a control plant.

In certain embodiments, the genomic locus that encodes an endogenous MFT polypeptide comprises a polynucleotide sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 7.

As used herein “increased expression” refers to any detectable increase in the level of the encoded polypeptide as compared to a control plant (e.g., non-modified plant). Similarly, as used here “decreased expression” refers to any detectable decrease in the level of the encoded polypeptide as compared to a control plant (e.g., non-modified plant). The level of expression can be measure using routine methods known in the art such as Western blotting, mass spectrometry, and ELISA.

In certain embodiments, the targeted genetic modification is selected from the group consisting of an insertion, deletion, single nucleotide polymorphism (SNP), and a polynucleotide modification. In certain embodiments, the targeted genetic modification is present in (a) the coding region; (b) a non-coding region; (c) a regulatory sequence; (d) an untranslated region; or (e) any combination of (a)-(d) of the genomic locus that encodes the MFT polypeptide.

In certain embodiments the DNA modification increasing the level and or activity of the MFT polypeptide is an insertion of one or more nucleotides, preferably contiguous, in the genomic locus. For example, the insertion of an expression modulating element (EME), such as an EME described in PCT/US2018/025446 (WO2018183878), in operable linkage with the MFT gene. In certain embodiments, the targeted DNA modification may be the replacement of the endogenous MFT promoter with another promoter known in the art to have higher expression. In certain embodiments, the targeted DNA modification may be the insertion of a promoter known in the art to have higher expression into the 5′UTR so that expression of the endogenous MFT polypeptide is controlled by the inserted promoter. In certain embodiments, the DNA modification is a modification to optimize Kozak context to increase expression. In certain embodiments, the DNA modification is a polynucleotide modification or SNP at a site that regulates the stability of the expressed protein.

In certain embodiments the DNA modification decreasing the level and or activity of the MFT polypeptide is an MFT gene knockout. In certain embodiments, the targeted DNA modification may be the replacement of the endogenous MFT promoter with another promoter known in the art to have lower expression. In certain embodiments, the targeted DNA modification may be the insertion of a promoter known in the art to have lower expression into the 5′UTR so that expression of the endogenous MFT polypeptide is controlled by the inserted promoter. In certain embodiments, the DNA modification is a polynucleotide modification or SNP at a site that regulates the stability of the expressed protein.

C. Breeding Method for Increasing Seed Oil and/or Protein Content

Further provided herein are methods of producing a seed having increased protein and/or oil content comprising a polynucleotide encoding a polypeptide that is at least 80% identical to any one of SEQ ID NOs: 2, 10, 12, 14, 16, 18, 20, or 22, the polypeptide comprising a modification at a position other than the amino acid corresponding to L106 in SEQ ID NO: 2 that increases oil content in the first plant with a second different plant line and harvesting the seed produced thereby. In certain embodiments, the harvested seed comprises the polynucleotide.

In certain embodiments, the first plant line comprises a polynucleotide sequence that is at least 97%, 98%, or 99% identical to SEQ ID NO: 7. In certain embodiments, the second plant line comprises a nucleotide sequence that is at least 97%, 98%, or 99% identical to SEQ ID NO: 8. In certain embodiments, the seed harvested in the method comprises a polynucleotide sequence that is at least 97%, 98%, or 99% identical to SEQ ID NO: 7.

The modification that increases the oil content may be any modification described herein, such as a substitution of a serine at the amino acid residue corresponding to position L140 of SEQ ID NO: 2, or any modification known in the art to increase oil content.

In certain embodiments, the second plant line comprises a nucleotide sequence encoding an amino acid sequence that is at least 80% identical to any one of SEQ ID NOs: 2, 10, 12, 14, 16, 18, 20, or 22. In certain embodiments, the second plant comprises a non-leucine at the amino acid residue corresponding to L106 of SEQ ID NO: 2.

Thus, in certain embodiments, the method comprises crossing a first plant line comprising a polynucleotide encoding a polypeptide that is at least 80% identical to any one of SEQ ID NOs: 2, 10, 12, 14, 16, 18, 20, or 22 and comprising a leucine at the amino acid residue corresponding to L106 of SEQ ID NO: 2 and a modification that increases the oil content in the first plant with a second plant line comprising a nucleotide sequence encoding an amino acid sequence that is at least 80% identical to any one of SEQ ID NOs: 2, 10, 12, 14, 16, 18, 20, or 22 and harvesting the seed produced thereby.

In certain embodiments, the method further comprises growing the seed to produce a second-generation progeny plant that comprises the polypeptide and backcrossing the second-generation progeny plant to the second plant to produce a backcross progeny plant that comprises the polypeptide and produces backcrossed seed with increased oil content.

The increase in seed oil and/or protein may be any increase described herein. In certain embodiments, the seed has a modified amount of fatty acids as described herein. In certain embodiments, the plants have a yield that is greater than or within 0.5%, 1%, 1.5%, 2%, 2.5%, 3%, 3.5%, 4%, 4.5%, or 5%, as compared to the corresponding control plant.

The following are examples of specific embodiments of some aspects of the invention. The examples are offered for illustrative purposes only and are not intended to limit the scope of the invention in any way.

### Example 1

This example demonstrates the generation and characterization of the modified MFT polypeptide to increase oil and/or protein content.

To provide a novel high protein and oil source for breeding, a gamma-ray mutagenized population was created and a high oil and protein mutant, HiPO #538 was identified using an integrated screening method. In 2018 in an Iowa field, M4 HiPO #538 mutant and WT plants were grown in 3 short rows. Seed harvested from the field were analyzed by wet chemistry for oil and protein content as described previously (WO 2018/160485 A1). Average oil and protein content of 3 replicates is shown in Table 2. HiPO #538 mutant showed a 2.8-point increase in oil and 0.9-point increase in protein compared to WT at 13% grain moisture. In 2019, M5 HiPO #538 mutant sublines were tested in 7 locations in Midwest US. Grain samples from 5 locations with 2 replicates per location were analyzed for oil and protein content by wet chemistry. On average, HiPO #538 mutant showed a 2.9-point increase in oil and 1.2-point increase in protein, which is consistent with the results from 2018 field test (Table 2—seed oil and protein content is adjusted to 13% seed moisture content and * indicates HiPO #538 shows a significant increase in oil and protein contents compared to wild type at p<0.05; as determined by Student's t-test). Overall, HiPO #538 increases seed protein+oil by 3.7-4 points with no inverse correlation between protein and oil in 2-year field trials. Fatty acid profiling was also determined in 2018 grain by gas chromatography. HiPO #538 did not show a significant change in fatty acid composition compared to WT (Table 3).

TABLE 2
Seed oil and protein content (13% moisture
content) of HiPO#538 mutant and wild type

| | 2018 Johnston field | | | 2019 multi-locations | | |
|---|---|---|---|---|---|---|
| | WT | HiPO#538 | Diff | WT | HiPO#538 | Diff |
| Seed oil % | 18.7 | 21.5 | 2.8* | 18.5 | 21.4 | 2.9* |
| Seed protein % | 35.4 | 36.3 | 0.9 | 34.5 | 35.7 | 1.2* |
| Protein + oil % | 54.1 | 57.8 | 3.7* | 53.0 | 57.1 | 4.0* |

TABLE 3
Fatty acid composition (relative %) of HiPO#538 mutant and wild type

| | Palmitic | Stearic | Oleic | | Linolenic |
|---|---|---|---|---|---|
| | acid (16:0) | acid | acid | Linoleic acid | acid |
| | % | (18:0) % | (18:1) % | (18:2) % | (18:3) % |
| WT | 10.1 | 3.6 | 22.4 | 54.5 | 8.12 |
| HiPO#538 | 10 | 3.9 | 20.8 | 57.3 | 6.81 |

Soybean seed protein content has reduced gradually with increasing grain yield through breeding. Breeding for high yield is associated with decreased protein. To determine if HiPO #538 affects grain yield, 12 M4 sublines derived from HiPO #538 mutant were tested in 7 locations with 2 replicates per location. Early stand count, vigor and maturity were scored in the field. None of the 12 mutant sublines show a significant difference, as determined by Student's t-test, in grain yield or plant height compared to wild type (Table 4). In addition, the HiPO #538 mutant also did not show any difference from wild type in early stand count and plant vigor. HiPO #538 mutant, however, matured 2-3 days earlier than the wild type, which is a favorable trait for breeding.

TABLE 4
HiPO#538 mutant yield trials

| | | Plant | |
|---|---|---|---|
| | Yield | height | Maturity |
| | (bu/a) | (in) | (Days) |
| WT | 58.1 | 39.5 | 123.1 |
| HiP#O538 subline 1 | 57.3 | 39.5 | 120.7* |
| HiP#O538 subline 2 | 56.0 | 37.9 | 119.9* |
| HiP#O538 subline 3 | 59.9 | 40.1 | 120.9* |
| HiP#O538 subline 4 | 59.3 | 39.8 | 120.2* |
| HiP#O538 subline 5 | 59.2 | 39.9 | 122.7* |
| HiP#O538 subline 6 | 60.3 | 39.1 | 120.5* |
| HiP#O538 subline 7 | 59.2 | 41.2 | 120.4* |
| HiP#O538 subline 8 | 62.2 | 39.6 | 122.2* |
| HiP#O538 subline 9 | 57.6 | 40.4 | 120.7* |
| HiP#O538 subline 10 | 61.7 | 39.8 | 122.8* |
| HiP#O538 subline 11 | 59.2 | 39.1 | 121.6* |
| HiP#O538 subline 12 | 57.9 | 38.3 | 120.1* |

*indicates HiPO#538 shows a significant early in maturity compared to wild type at p < 0.05; as determined by Student's t-test

These data demonstrate that the HiPO #538 mutant line has increased protein and oil content with no significant difference in yield as compared to a control line

### Example 2

This example demonstrates the identification and validation of the causative mutations for HiPO #538.

To identify the causative mutation for high protein and oil, DNA was isolated from 3 sublines of the HiPO #538 mutant and was subjected to whole-genome sequencing on the Illumina platform. Raw Illumina reads produced for each sequenced subline were processed using custom internal scripts (SNPfinder pipeline) which performs read mapping and detection of sequence variants (specifically single nucleotide polymorphisms (SNPs) or short Insertions or deletions (InDels) (˜50 bp or less). In addition to identifying SNPs and short InDels, the Illumina sequencing data were also analyzed using custom internal pipelines to identify large deletions (greater than 500 bp) in the genomic sequence of the soy mutant plants. Compared to a wild type reference genome, 150 mutant specific SNPs and a ˜1 kb deletion in an intronic region were identified. Among 150 SNPs, only 5 reliable genes contain an amino acid change as shown in Table 5. Other SNPs are either in intronic region or in genic region without affecting the amino acid residue.

TABLE 5
Non-synonymous mutations identified in HiPO#538 mutant

| Gene | SNP | Function annotation |
|---|---|---|
| Glyma.05g244100.1 | non-synonymous | PEBP family protein |
| Glyma.09g038300.1 | non-synonymous | TELO2-interacting protein |
| | | 1 like |
| Glyma.01g038300.1 | non-synonymous | SIN3-like 2%2C putative |
| | | isoform 2 |
| Glyma.U018800/ | non-synonymous | Putative yl1 nuclear protein |
| Glyma11g16030 | | |
| Glyma.18g143700.2 | non-synonymous | MATE efflux family protein |

Among the 5 genes containing missense mutations, only glyma.05g244100 shows a seed specific expression pattern during seed development, which is consistent with oil and protein accumulation (Table 6).

TABLE 6
Expression of MFT gene (glyma.05g244100) in soybean

| | Gene Expression |
|---|---|
| Samples | (ppm) |
| soy_embryogenic_suspension_culture (cell culture) | 125.6 |
| soy_cotyledons (cotyledon) | 19.1 |
| soy_somatic_embryos_germination (embryo) | 101.4 |
| soy_somatic_embryos_dry_down (embryo) | 21.5 |
| soy_somatic_embryos_maturation_SHAM | 5279.9 |
| (embryo) | |
| soy_somatic_embryos_maturation (embryo) | 636.1 |
| soy_flower (flower) | 0.0 |
| soy_flower_cluster (flower) | 17.2 |
| soy_leaf_flowering (leaf) | 12.6 |
| soy_leaf_first_trifolate (leaf) | 6.9 |
| soy_shoot_apical_meristem (meristem) | 3.1 |
| soy_leaflet_petiole (petiole) | 0.9 |
| soy_main_petiole (petiole) | 0.0 |
| soy_pods_1cm (pod) | 1.4 |
| soy_pods_2cm (pod) | 1.4 |
| soy_root_seedling (root) | 20.2 |
| soy_root_tips_seedling (root) | 0.4 |
| soy_seed_50_DAF (seed) | 356.9 |
| soy_seed_30_DAF (seed) | 5522.9 |
| soy_seed_15_DAF (seed) | 1997.6 |
| soy_seed_50DAF (seed) | 786.9 |
| soy_stem (stem) | 0.0 |

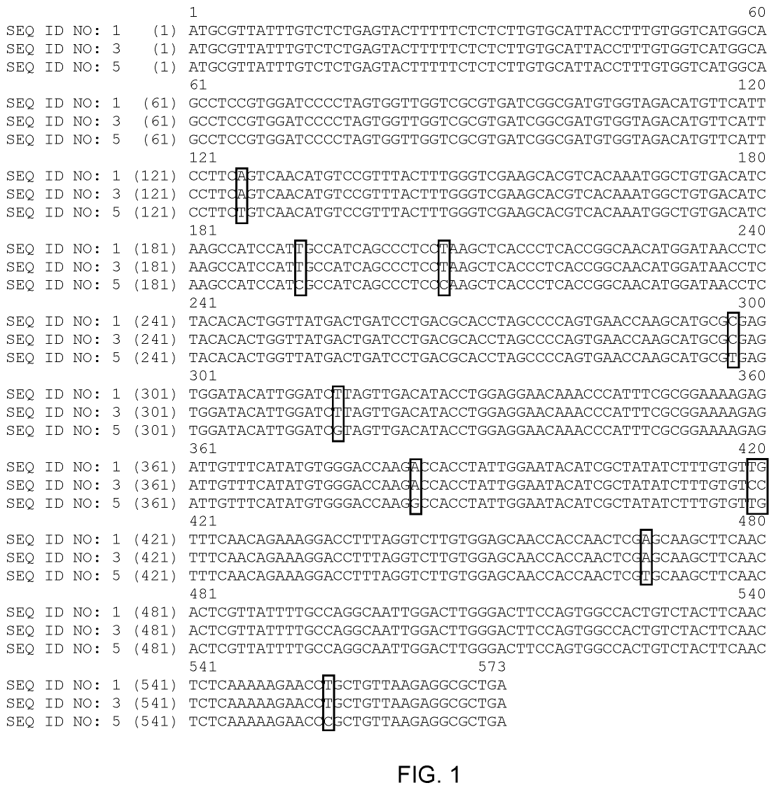

Glyma.05g244100 encodes a Mother of FT (flowering time) and TFL1 (terminated flowering locus1) (MFT)-like protein, which is a member of the phosphatidylethanolamine binding protein (PEBP) family. Compared to wild type MFT, MFT from HiPO #538 contains two base pair changes, which lead to a single amino change from a leucine residue to a serine as shown in FIG. 1 (polynucleotide) and FIG. 2 (amino acid).

To confirm that MFT is the causative mutation, HiPO #538 was crossed to a wild type soybean to produce a F2 mapping population. Approximately 800 F2 plants were genotyped for the SNP in the MFT gene. Protein and oil content of F3 seeds collected from F2 plants were determined by FT-NIR. All F2 plants homozygous for the MFT mutation showed a high oil phenotype similar to HiPO #538 mutant while all F2 plants with wild type MFT showed a normal oil phenotype similar to the wild type parent. F2 plants heterozygous for MFT mutation showed an oil level at the midpoint of two parents, suggesting that the mutant is semi-dominant (Table 7). The single amino acid mutation in the MFT gene shows a strong co-segregation with seed oil content. Since there were no other mutations in a 2 cM region flanking the MFT gene detected by whole genome sequencing, it was concluded that MFT is the causative mutation for the high oil and protein content in HiPO #538.

These results demonstrate that the leucine to serine substitution at position 140 of MFT is a causative mutation for high oil and protein.

TABLE 7
Co-segregation of MFT mutation and high oil in the F2 population

| | Seed oil % | Seed protein % | # of plants |
|---|---|---|---|
| Parent-1, WT | 18.8 | 35.9 | 34 |
| Parent-2, HiPO#538 mutant | 21.1 | 35.8 | 28 |
| F2 mft/mft homo mutant plant | 20.4 | 36.4 | 93 |
| F2 MFT/mft het mutant plant | 19.5 | 36.2 | 284 |
| F2 MFT/MFT WT plant | 18.8 | 36.1 | 134 |

### Example 3

This example demonstrates the characterization of MFT

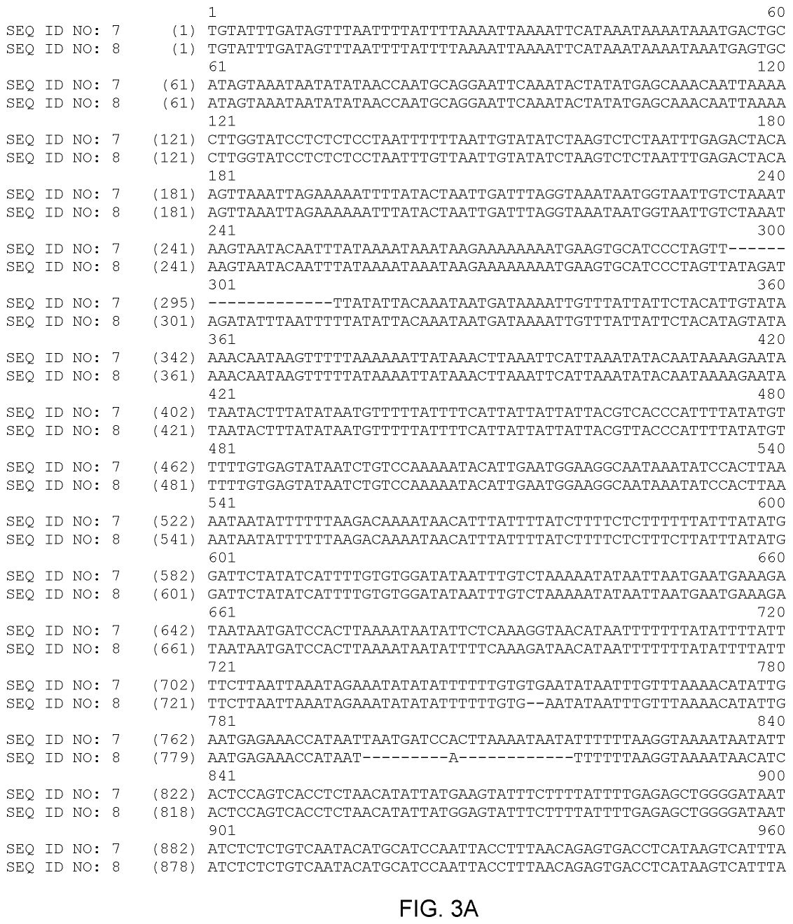

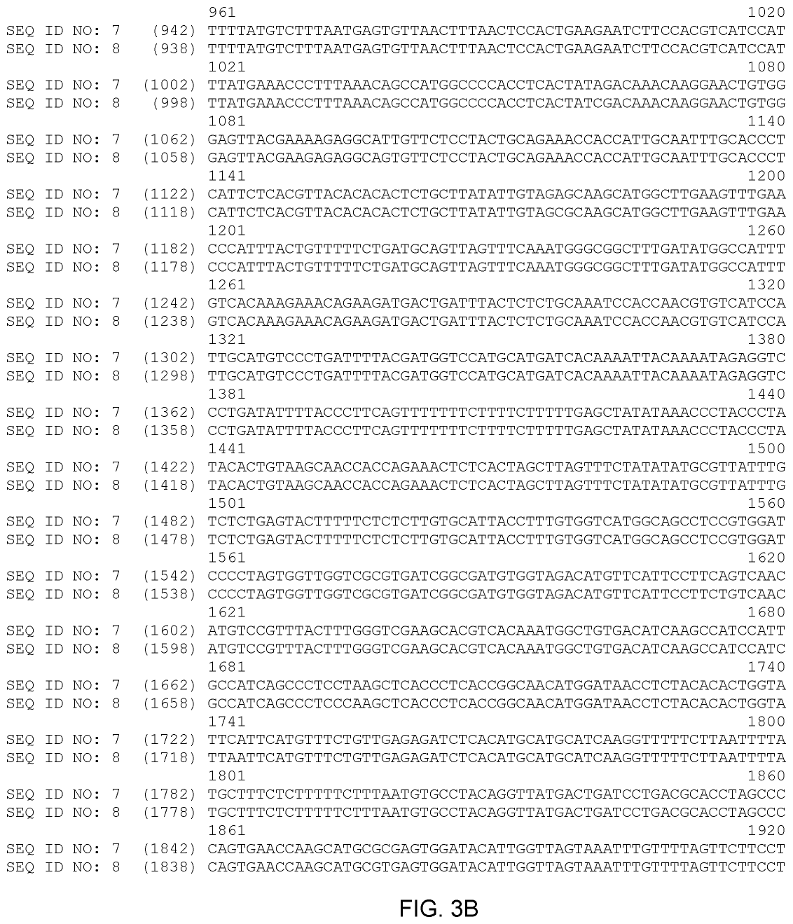

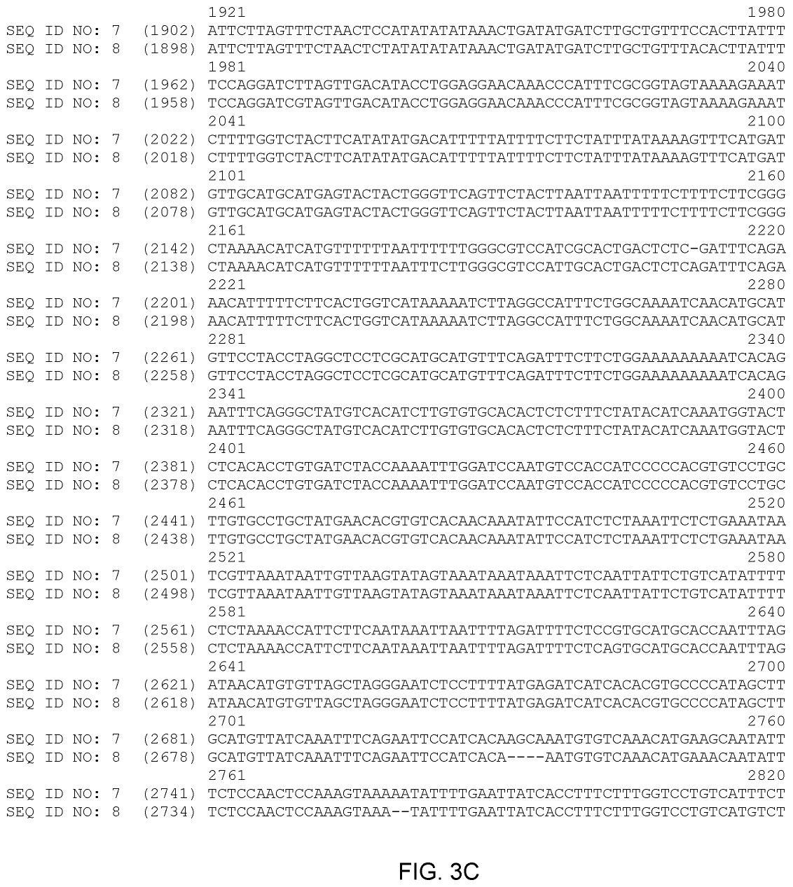

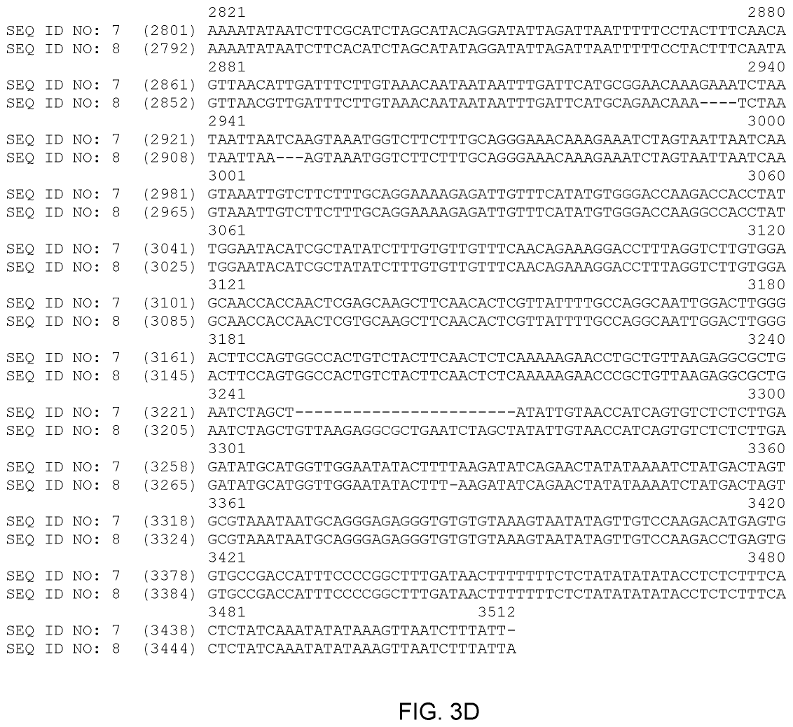

The MFT gene on chromosome 5 (glyma.05g244100) is located at 38.4 Mb which is within the interval of the major oil QTL and is associated strongly with seed oil content in a genome-wide association mapping study (Li et al 2018 Plant science 266:95). As described above, a single amino acid mutation in HiPO #538 increases seed oil content in an elite soybean background, suggesting MFT is a strong candidate underlying the QTL on chromosome 5. Wild soybean, Glycine soja, shows a low seed oil and high seed protein content. During domestication and breeding of soybean, seed oil content increases significantly from 12% in Glycine soja to 20% in current elite lines. To validate that MFT is responsible for oil increase during domestication, the MFT allele was isolated from wild soybean, Glycine soja line PI468916. Compared to the MFT allele from Glycine soja P1468916, multiple SNPs and small deletions/addition were found in the promoter and coding sequence in MFT isolated from an elite soybean line, which could be the causative mutations resulting in high oil in the elite line (FIGS. 3A-3D). Compared to MFT protein from Glycine soja, there is only a single amino acid change from a valine to leucine at position 106 (106 L), which could be a causative mutation for increasing oil in the elite soybean (FIG. 2). If an elite line still contains Glycine soja allele, selection of V106L allele should increase seed oil content in elite.

To validate that MFT is responsible for the high oil QTL on Chromosome 5, a frame-shift MFT knockout line has been generated by CRISPR/Cas9 editing which will allow for the determination of the function of MFT in the knockout line. The Glycine soja low oil MFT allele and normal oil MFT allele from an elite line will be introduced back to the MFT knockout line to determine the effect of 2 alleles on seed oil and protein content.

### Example 4

The example demonstrates the identification and characterization of MFT homologs from other crops.

To identify MFT homologs from other major crops the soybean MFT coding sequence (Glyma.05g244100.1) were used to query a combination of proprietary and public datasets using BLAST® (Basic Local Alignment Search Tool). Pairwise alignments of both nucleotide and amino acid sequences were completed using VNTI sequence alignment software to determine percent identity. Sequence ID's, crop species names and common names along with annotation identity are cataloged in Table 8. Nucleotide identity from different crop species range from around 60-80% with amino acid identity levels ranging from 60-85%.

TABLE 8
Nucleotide and amino acid identity levels of soybean MFT crop
homologs

| | | | % NT | % AA |
|---|---|---|---|---|
| SEQ ID | Species | Common name | identity | identity |
| 1, 2 | Glycine max | soybean | | |
| 9, 10 | Brassica napus | canola | 69.7 | 74.7 |
| 11, 12 | Gossypium | cotton | 72.3 | 77.5 |
| | raimondii | | | |
| 13, 14 | Zea maize | corn | 62.0 | 58.0 |
| 15, 16 | Triticum asestivum | wheat | 62.6 | 60.2 |
| 17, 18 | Medicago truncatula | alfalfa | 79.1 | 84.5 |
| 19, 20 | Oryza sativa | rice | 61.8 | 61.0 |
| 21, 22 | Sorghum bicolor | sorghum/great | 61.5 | 59.5 |
| | | millet | | |

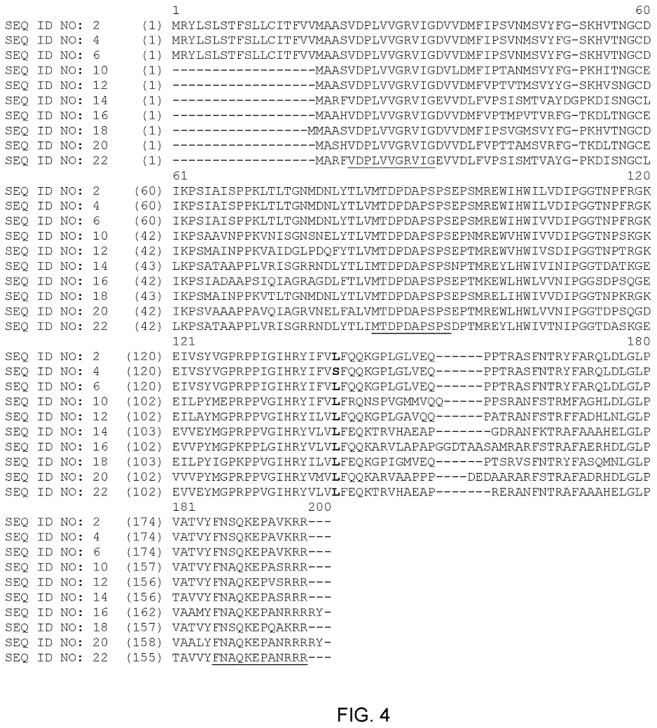

FIG. 4 shows an amino acid sequence alignment of MFT proteins from different crops. Three domains, VDPLVVGRVIG, MTDPDAPSPS, YFNXQKEPXXXRR, are highly conserved in all MFT proteins. The mutated leucine residue in HiPO #538 is conserved in all MFT genes.

To test if the modification of the leucine residue improves seed composition in other crops, the leucine residue in other crop MFT proteins will be changed to a serine residue as in HiPO #538 by CRISPR/Cas 9 editing. Change of leucine residue to other amino acid, such as threonine, could also improve seed composition in other crops.

### Example 5

This example demonstrates increasing seed protein and oil content by editing the MFT gene.

For genome engineering applications, the type II CRISPR/Cas system minimally requires the Cas9 protein and a duplexed crRNA/tracrRNA molecule or a synthetically fused crRNA and tracrRNA (guide RNA) molecule for DNA target site recognition and cleavage (Gasiunas et al. (2012) Proc. Natl. Acad. Sci. USA 109: E2579-86, Jinek et al. (2012) Science 337:816-21, Mali et al. (2013) Science 339:823-26, and Cong et al. (2013) Science 339:819-23). Described herein is a guideRNA/Cas endonuclease system that is based on the type II CRISPR/Cas system and consists of a Cas endonuclease and a guide RNA (or duplexed crRNA and tracrRNA) that together can form a complex that recognizes a genomic target site in a plant and introduces a double-strand -break into said target site.

To use the guide RNA/Cas endonuclease system in soybean, the Cas9 gene from Streptococcus pyogenes M1 GAS (SF370) was soybean codon optimized per standard techniques known in the art. To facilitate nuclear localization of the Cas9 protein in soybean cells, Simian virus 40 (SV40) monopartite amino terminal nuclear localization signal and Agrobacterium tumefaciens bipartite VirD2 T-DNA border endonuclease carboxyl terminal nuclear localization signal were incorporated at the amino and carboxyl-termini of the Cas9 open reading frame, respectively. The soybean optimized Cas9 gene was operably linked to a soybean constitutive promoter such as the strong soybean constitutive promoter GM-EF1A2 (US Patent Application Publication 2009/0133159) or regulated promoter by standard molecular biological techniques.

The second component to form a functional guide RNA/Cas endonuclease system for genome engineering applications is a duplex of the crRNA and tracrRNA molecules or a synthetic fusing of the crRNA and tracrRNA molecules, a guide RNA. To confer efficient guide RNA expression (or expression of the duplexed crRNA and tracrRNA) in soybean, the soybean U6 polymerase III promoter and U6 polymerase III terminator were used.

Plant U6 RNA polymerase III promoters have been cloned and characterized from Arabidopsis and Medicago truncatula (Waibel and Filipowicz, NAR 18:3451-3458 (1990); Li et al., J. Integrat. Plant Biol. 49:222-229 (2007); Kim and Nam, Plant Mol. Biol. Rep. 31:581-593 (2013); Wang et al., RNA 14:903-913 (2008)). Soybean U6 small nuclear RNA (snRNA) genes were identified herein by searching public soybean variety Williams82 genomic sequence using Arabidopsis U6 gene coding sequence. Approximately 0.5 kb genomic DNA sequence upstream of the first G nucleotide of a U6 gene was selected to be used as a RNA polymerase III promoter, for example, GM-U6-13.1 promoter or GM-U6-9.1 promoter, to express guide RNA to direct Cas9 nuclease to designated genomic site. The guide RNA coding sequence was 76 bp long and comprised a 20 bp variable targeting domain from a chosen soybean genomic target site on the 5′ end and a tract of 4 or more T residues as a transcription terminator on the 3′ end. The first nucleotide of the 20 bp variable targeting domain was a G residue to be used by RNA polymerase III for transcription. Other soybean U6 homologous genes promoters were similarly cloned and used for small RNA expression.

Since the Cas9 endonuclease and the guide RNA need to form a protein/RNA complex to mediate site-specific DNA double strand cleavage, the Cas9 endonuclease and guide RNA must be expressed in same cells. To improve their co-expression and presence, the Cas9 endonuclease and guide RNA expression cassettes were linked into a single DNA construct.

As described above, the Glyma.05g244100 encodes a Mother of FT and TFL1-like protein (MFT), which is a member of phosphatidylethanolamine binding protein (PEBP) family. To examine and validate the functions of Glyma.05g244100, a guide RNA (GM-MFT-CR1, GACCACTAGGGGATCCACGG, SEQ ID. 23) was designed in the exon1 of the gene to create frameshift mutations. Four frame shift (FS) variants, E1.2A, E1.5A, E1.10A and E1.8A, were generated. Homozygous frame shift T1 plants were identified. T2 seeds of 4 variants showed a significant increase in seed oil and protein content and a significant reduction in total carbohydrate (Table 9). This result demonstrates that MFT is the causative gene for high oil and protein in HiPO #538.

TABLE 9
T2 seed composition of MFT frame shift variants

| | | | | Seed | |
|---|---|---|---|---|---|
| | MFT | Seed | Seed | Protein + | Total |
| Variant | genotype | protein % | oil % | Oil % | carbohydrate % |
| 93Y21 | WT | 34.0 | 20.8 | 54.8 | 9.7 |
| E1.2A | homo FS | 35.7* | 21.4 | 57.1* | 8.3* |
| E1.5A | homo FS | 36* | 21.3 | 57.3* | 8.1* |
| E1.10A | homo FS | 35.8* | 21.1 | 56.9* | 8.4* |
| E1.8A | homo FS | 35.3* | 21.7 | 56.9* | 8.3* |

seed protein and oil % are based on 13% seed moisture.
*indicates variants show a significant change with P < 0.01 (T test) compared to 93Y21 wild type seeds

In addition, by designing another gRNA targeting the leucine residue of the MFT protein and by providing a donor template with the desirable nucleotide changes, the leucine to serine amino acid substitution in the endogenous MFT protein, as in the HiPO #538 mutant, will be generated by homology-mediated double strand break repair process. Furthermore, this leucine residue can be changed to other amino acid to improve MFT function and increase seed value in soybean and other crops by the base-editing technology (Ress, H. A. and Liu, D., 2018 Nature Reviews Genetics, 19, 770-788) or the prime editing technology (Anzalone et.al., 2019 Nature, 576, 149-157).

### Example 6

This example demonstrates increasing seed protein and oil content by modifying MFT gene expression.

To further increase seed oil and protein content, expression of the MFT allele can be driven by a strong soybean seed specific promoter, such as Gm-Ole 2b promoter or Gm-Slb1 promoter, and a soybean terminator, such as Gm-MYB2 terminator. The expression vectors containing constructs such as listed in Table 10 can be introduced into soybean by the method of particle gun bombardment (Klein et al., Nature (London) 327:70-73 (1987); U.S. Pat. No. 4,945,050) using a BIORAD Biolistic PDS1000/He instrument or Agrobacteria or Ochrobacteria transformation. Transgenic seed oil and protein content will be determined by SS-NIR and FT-NIR spectroscopy as described previously (Roesler et al Plant Physiol. 2016 878-893). Increased expression of the MFT allele should increase oil and protein.

TABLE 10
List of constructs to be used for transgenic expression

| Promoter | Gene | Terminator |
|---|---|---|
| Gm-Ole2b promoter | Gm-MFT allele from WT | Gm-MYB2 Term |
| Gm-Ole2b promoter | Gm-MFT allele from HiPO#538 | Gm-MYB2 Term |
| Gm-Slb1 promoter | Gm-MFT allele from WT | Gm-MYB2 Term |
| Gm-Slb1 promoter | Gm-MFT allele from HiPO#538 | Gm-MYB2 Term |

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1-50. (canceled)

51. A soybean cell having increased oil content and comprising decreased expression or stability of an endogenous MFT polypeptide comprising an amino acid sequence that is at least 95% identical to SEQ ID NO: 2 when compared with a control soybean cell.

52. The soybean cell of claim 51, wherein the soybean cell comprises a modified MFT gene regulatory element sequence and decreased expression of the endogenous MFT polypeptide.

53. The soybean cell of claim 51, wherein the soybean cell comprises a modified MFT gene coding sequence such that the expression or stability of the endogenous MFT polypeptide is decreased.

54. The soybean cell of claim 53, wherein the modified MFT gene coding sequence encodes a modified MFT polypeptide sequence comprising one or more of an amino acid deletion, an amino acid insertion, an amino acid substitution, or any combination thereof as compared to SEQ ID NO: 2.

55. The soybean cell of claim 54, wherein the modified MFT polypeptide sequence comprises a non-leucine at the amino acid residue corresponding to position L140 of SEQ ID NO: 2.

56. The soybean cell of claim 55, wherein the modified MFT polypeptide sequence comprises a glycine, asparagine, glutamine, alanine, serine, cysteine, or threonine at the amino acid residue corresponding to position L140 of SEQ ID NO: 2.

57. The soybean cell of claim 51, wherein the soybean cell comprises a knockout of an endogenous MFT gene.

58. A soybean plant comprising the soybean cell of claim 51.

59. A soybean seed comprising the soybean cell of claim 51.

60. The soybean seed of claim 59, wherein the oil content of the soybean seed is increased by at least a 1 percentage point, the protein content of the soybean seed is increased by at least a 0.25 percentage point, or a combination thereof, as compared to a control soybean seed when measured at 13% moisture content.

61. A soybean plant comprising soybean seeds having increased oil content as compared with control seeds of a control plant when measured at 13% seed moisture content, the soybean plant comprising decreased expression or stability of an endogenous MFT polypeptide comprising an amino acid sequence that is at least 95% identical to SEQ ID NO: 2 as compared with the control plant.

62. The soybean plant of claim 61, wherein the soybean seeds further comprise at least at least a 1 percentage point increase in oil content, a 0.25 percentage point increase in protein content, or a combination thereof, as compared to the control seeds when measured at 13% moisture content.

63. The soybean plant of claim 61, wherein the soybean plant comprises a modified MFT gene regulatory element sequence and decreased expression of the endogenous MFT polypeptide.

64. The soybean plant of claim 61, wherein the soybean plant comprises a modified MFT gene coding sequence such that the expression or stability of the endogenous MFT polypeptide is decreased.

65. The soybean plant of claim 64, wherein the modified MFT gene coding sequence encodes a modified MFT polypeptide sequence comprising one or more of an amino acid deletion, an amino acid insertion, or an amino acid substitution, or any combination thereof as compared to SEQ ID NO: 2.

66. The soybean plant of claim 65, wherein the modified MFT polypeptide sequence comprises a non-leucine at the amino acid residue corresponding to position L140 of SEQ ID NO: 2.

67. The soybean plant of claim 61, wherein the soybean plant comprises a knockout of an endogenous MFT gene

68. A method of producing the soybean plant of claim 61,
the method comprising introducing into an endogenous MFT gene a modification decreasing the expression or stability of the endogenous MFT polypeptide.

69. A method of producing a seed having increased oil content, the method comprising:
(a) crossing a first plant line comprising decreased expression or stability of an endogenous MFT polypeptide comprising an amino acid sequence that is at least 95% identical to SEQ ID NO: 2 and comprising an increased oil content in a seed of the plant as compared with a control plant with a second different plant line; and
(b) harvesting the seed produced thereby.

70. The method of claim 69, wherein the first plant line comprises a targeted DNA modification that results in a knockout of an endogenous MFT gene or that introduces into the endogenous MFT gene one or more of a nucleotide insertion, a nucleotide deletion, a single nucleotide polymorphism (SNP), or any combination thereof.
